(19) United States
(12) Patent Application Publication
(10) Pub. No.: US 2024/0287564 A1
(43) Pub. Date: Aug. 29, 2024

(54) ACHOLETIN BIOPOLYMERS AND METHODS FOR ENZYMATIC SYNTHESIS

(71) Applicant: THE UNIVERSITY OF BRITISH COLUMBIA, Vancouver (CA)

(72) Inventors: Stephen G. Withers, Vancouver (CA); Spencer S.S. MacDonald, Vancouver (CA)

(73) Assignee: THE UNIVERSITY OF BRITISH COLUMBIA, Vancouver, BC (CA)

(21) Appl. No.: 18/567,004

(22) PCT Filed: Jun. 7, 2022
(86) PCT No.: PCT/CA2022/050907

§ 371 Date: Dec. 4, 2023

Related U.S. Application Data
(60) Provisional application No. 63/197,820, filed on Jun. 7, 2021.

(51) Int. Cl. C12P 19/04; C12N 9/10; C12N 9/12; C12N 9/42; C12P 19/18; C12P 19/26

(52) U.S. Cl. CPC C12P 19/04; C12N 9/1051; C12N 9/1205; C12N 9/2442; C12P 19/18; C12P 19/26; C12Y 204/01211; C12Y 207/01; C12Y 302/01014

(57) ABSTRACT

Provided herein are β-1,3-linked biopolymers (acholetin polysaccharides). Furthermore, provided herein are enzymatic methods and systems for producing β-1,3-linked oligosaccharides and polysaccharides using β-1,3-N-acetylglucosaminide phosphorylase (Acholetin phosphorylase (AchP)). The AchP was sourced from the genome of the cell wall-less Mollicute bacterium, Acholeplasma laidlawii and was found to synthesize β-1,3-linked N-acetylglucosamine (GlcNAc) or N-acetylgalactosamine (GalNAc) oligomers using the donor. α-N-acetylglucosamine 1-phosphate (GlcNAc1-P) or N-acetylgalactosamine 1-phosphate (GalNAc1-P).

### CROSS REFERENCE TO RELATED APPLICATION

This application claims the benefit of U.S. Provisional Patent Application Ser. No. 63/197,820 filed on 7 Jun. 2021, entitled “ACHOLETIN BIOPOLYMERS AND METHODS FOR ENZYMATIC SYNTHESIS”.

### TECHNICAL FIELD

The present invention relates to the field of enzyme compositions. In particular, the invention relates to β-1,3-N-acetylglucosaminide phosphorylase (Acholetin phosphorylase (AchP)), and providing enzymatic methods and systems for producing β-1,3-linked biopolymers (i.e. oligosaccharides and polysaccharides).

### BACKGROUND

Polysaccharides are the most abundant biopolymers on earth, play numerous key roles in living systems and have been utilized to develop an extensive range of functional materials for human use¹. The renewability and carbon neutrality of bio-sourced polysaccharides has led to significant interest in their potential to replace synthetic polymers, such as plastics, derived from fossil fuels². Beyond being sustainable and environmentally friendly, poly- and oligo-saccharides have seen considerable biomedical applications due to their favorable biocompatible and biodegradable properties³⁴. They have been central in the development of nano- and micro-particles for drug delivery systems⁵⁻¹¹, glycan conjugated therapeutics¹²⁻¹⁴, wound dressings¹⁵⁻¹⁹ and scaffolds for tissue engineering and 3D bioprinting²⁰⁻²⁶. The diverse applications of poly- and oligo-saccharides originate from the identities of their monomeric precursors and the types of glycosidic linkages that connect them. Together with the gamut of potential precursors and the array of possible linkages, carbohydrates are able to adopt a wide range of structures and functions, with greater conceivable complexity than their amino and nucleic acid counterparts. With this added complexity comes added difficulty when attempting to chemically synthesize poly- or oligo-saccharides²⁷. In the biomedical context it is sometimes important that synthesis occurs under conditions that result in a uniform and sequence-defined carbohydrate to achieve the desired properties of the functional material. However, due in large part to the more or less chemical equivalency of individual glycoside hydroxyl groups, chemical synthesis often requires multiple inefficient protection and deprotection steps to control the stereo- and regio-specificity of glycosylation²⁸, resulting in increased costs and diminished yields. Efforts to chemically synthesize β-1,3-linked oligosaccharides have been limited and have only reported degrees of polymerization of 5 or 6^{75,76}. Enzymatic synthesis represents an advantageous alternative to achieving uniform and sequence-defined carbohydrates by exploiting an enzyme's innate substrate specificity and conformational control, thereby avoiding the inefficiencies associated with chemical synthesis^{28,29}.

Glycoside phosphorylases (GPs) are a class of Carbohydrate Active Enzymes (CAZymes)³⁰ that have seen frequent use for poly- and oligo-saccharide synthesis. GPs act through a process known as phosphorolysis that cleaves the glycosidic linkage with a phosphate molecule resulting in the release of a sugar 1-phosphate^{31,32}. Due to the roughly equivalent free energy associated with the inter-sugar glycosidic linkage and the glycosyl phosphate bond of the released sugar 1-phosphate, GPs can perform phosphorolysis in reverse³³. This allows GPs to be used for carbohydrate synthesis by adding glycosyl moieties from sugar 1-phosphates to suitable acceptors³⁴. Furthermore, this synthetic paradigm is amenable to large scale applications due to the relatively low cost associated with the sugar 1-phosphate starting materials. The innate substrate specificity and conformational control offered by GPs together with their inexpensive starting materials make these enzymes attractive tools for poly- and oligo-saccharide synthesis. As new GP activities are discovered and the array of these useful biocatalysts continues to grow, so too does the potential to generate novel and diverse carbohydrate-based materials.

### SUMMARY

The present invention is based in part, on the surprising discovery of the functional and structural characterization of a β-1,3-N-acetylglucosaminide phosphorylase, a previously uncharacterized glycosyl phosphorylase (GP) belonging to the CAZy family GH94. The GP was sourced from the genome of the cell wall-less Mollicute bacterium, Acholeplasma laidlawii and found to synthesize β-1,3-linked N-acetylglucosamine (GlcNAc) oligomers using the donor, α-N-acetylglucosamine 1-phosphate (GlcNAc1-P). The resulting biopolymer, poly-β-1,3-N-acetylglucosamine described herein as Acholetin phosphorylase (AchP). Furthermore, it completes the set of possible β-linked GlcNAc homo-polysaccharides together with (1) poly-β-1,4-N-acetyl-glucosamine, or chitin, the major component of arthropod exoskeletons, fungi cell walls and the second most abundant biopolymer on earth, and (2) poly-β-1,6-N-acetylglucosamine (PNAG), a key virulence factor required for biofilm formation by numerous pathogenic bacteria. Poly-β-1,3-N-acetylglucosamine was denoted, acholetin, a combination of the of the genus Acholeplasma and the well-known β-1,4-GlcNAc polysaccharide, chitin. Therefore, the new β-1,3-N-acetylglucosaminide phosphorylase is referred to, hereafter, as acholetin phosphorylase (AchP).

Acholetin phosphorylase (AchP) was discovered as part of a larger study aiming to characterize a phylogenomically diverse gene synthesis library (prepared by the Joint Genome Institute) of glycoside phosphorylases (GPs). A particular objective was to find phosphorylases that degrade chitin, since at present only N,N′-diacetylchitobiose phosphorylases are known. Such a chitin phosphorylase could be useful in large scale conversion of waste chitin into useful biochemicals. The broader study targeted GPs across five carbohydrate active enzyme (CAZy) families, but for the specific purpose noted we were most interested in members of GH94, the family that contains N,N′-diacetylchitobiose phosphorylases. To that end, activity-based screening was performed looking for phosphate release from the sugar phosphate donor in the presence of various acceptors; most importantly for this purpose GlcNAc and N,N′-diacetylchitobiose. Only one of the 18 GH94 enzymes screened yielded a strong hit with GlcNAc and this was also the only one to transfer to N,N′-diacetylchitobiose (FIG. 3). We therefore proceeded to investigate this lone hit in more detail, both through detailed informatics analysis and through kinetic and mechanistic studies on purified enzyme. These studies led to the fortuitous discovery of a never previously described β-1,3-linked biopolymer, methods to enzymatically synthesize said biopolymer and its uses.

In a first embodiment there is provided a polysaccharide, the polysaccharide including repeated monomers of N-acetyl-glucosamine (GlcNAc) or N-acetylgalactosamine (GalNAc) linked by glycosidic bonds having a β-configuration between the C1 position of the first GlcNAc or GalNAc ring and the C3 position of the adjacent GlcNAc or GalNAc ring, having the structure of Formula I

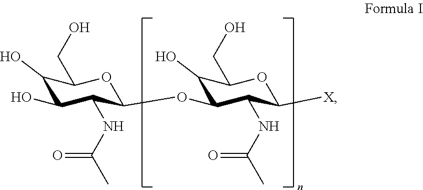

wherein, n may be an integer of 10 or greater; and X may be selected from —OH;

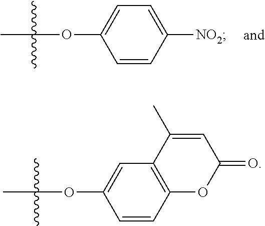

In a further embodiment there is provided a method of making an oligosaccharide or a polysaccharide, the method including enzymatic synthesis with a glycoside phosphorylase (GP) that links monomeric GlcNAc or GalNAc via a β-1,3-glycosidic linkage, wherein the oligosaccharide or the polysaccharide includes repeated monomers of GlcNAc or GalNAc linked by glycosidic bonds having a β-configuration between the C1 position of the first GlcNAc or GalNAc ring and the C3 position of the adjacent GlcNAc or GalNAc ring, may have the structure of Formula I

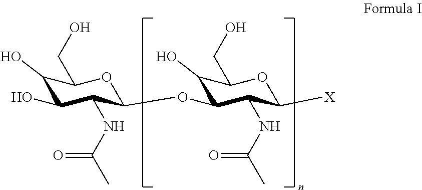

wherein, n may be an integer between 2 and 9, to form the oligosaccharide; n may be an integer of 10 or greater, to form the polysaccharide; and X may be selected from —OH;

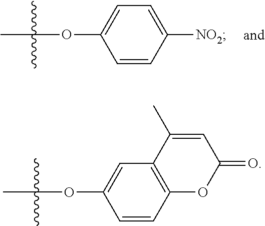

In a further embodiment there is provided a method of making an oligosaccharide or a polysaccharide, the method including: (a) generating a GlcNAc-1-P or GalNAc-1-P, as a glycosyl donor, by reacting an N-acetylhexosamine-1-kinase (NahK) with GlcNAc or GalNAc and ATP; (b) initiating a reverse phosphorolysis reaction by mixing the GlcNAc-1-P or GalNAc-1-P precipitate from step (a) with a glycosyl acceptor with a glycoside phosphorylase; wherein the oligosaccharide or the polysaccharide may include repeated monomers of GlcNAc or GalNAc may be linked by glycosidic bonds having a β-configuration between the C1 position of the first GlcNAc or GalNAc ring and the C3 position of the adjacent GlcNAc or GalNAc ring, may have the structure of Formula II

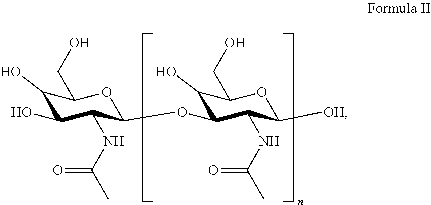

may be an integer between 2 and 9, to form the oligosaccharide; and n may be an integer of 10 or greater, to form the polysaccharide.

In a further embodiment there is provided a method of making an oligosaccharide or a polysaccharide, the method including: (a) generating a GlcNAc-1-P or GalNAc-1-P, as a glycosyl donor, by reacting an N-acetylhexosamine-1-kinase (NahK) with GlcNAc or GalNAc and ATP; (b) initiating a reverse phosphorolysis reaction by mixing the GlcNAc-1-P or GalNAc-1-P precipitate from step (a) with a glycosyl acceptor with a glycoside phosphorylase; wherein the oligosaccharide or the polysaccharide may include repeated monomers of GlcNAc or GalNAc may be linked by glycosidic bonds having a β-configuration between the C1 position of the first GlcNAc or GalNAc ring and the C3 position of the adjacent GlcNAc or GalNAc ring, may have the structure of Formula II

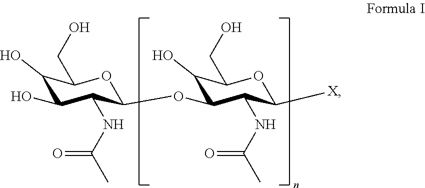

wherein, n may be an integer between 2 and 9, to form the oligosaccharide; n may be an integer of 10 or greater, to form the polysaccharide; and X may be selected from —OH;

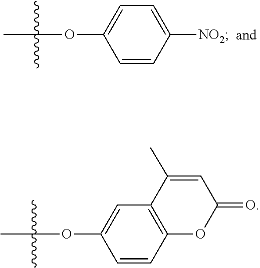 [SMILES: *Oc1ccc([N+](=O)[O-])cc1 ; "and" ; *Oc1ccc2oc(=O)cc(C)c2c1]

In a further embodiment there is provided a method of making an oligosaccharide or a polysaccharide, the method including enzymatic synthesis with a glycoside phosphorylase (GP) that links monomeric GlcNAc via a β-1,3-glycosidic linkage, wherein the oligosaccharide or the polysaccharide may include repeated monomers of N-acetyl-glucosamine linked by glycosidic bonds having a β-configuration between the C1 position of the first GlcNAc ring and the C3 position of the adjacent GlcNAc ring, having the structure of Formula I:

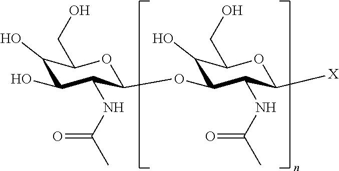

Formula I wherein, n may be an integer between 2 and 9, to form the oligosaccharide; n may be an integer of 10 or greater, to form the polysaccharide; and X may be selected from —OH;

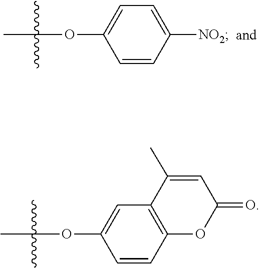 [SMILES: *Oc1ccc([N+](=O)[O-])cc1 ; "and" ; *Oc1ccc2oc(=O)cc(C)c2c1]

In a further embodiment there is provided a method of adding GlcNAc or GalNAc via a μ-1,3 linkage to a GlcNAc or GalNAc at the non-reducing end of an oligosaccharide, a polysaccharide, a chitin or a chito-oligosaccharide, the method including reverse phosphorolysis with a glycoside phosphorylase.

In a further embodiment there is provided a reaction composition, the reaction composition including at least: (a) a GlcNAc-1-P or GalNAc-1-P as a glycosyl donor; (b) GlcNAc or GalNAc as a glycosyl acceptor; and (c) a β-1,3-GlcNAc phosphorylase enzyme including an amino acid sequence that may be at least 95% identical to SEQ ID NO: 2, wherein the amino acid sequence has β-1,3-GlcNAc phosphorylase enzyme activity, and wherein the β-1,3-GlcNAc phosphorylase enzyme synthesizes a 3-1,3-glycosidic linkages between the donor and acceptor.

In a further embodiment there is provided a method of making an polysaccharide of any one of claims 1-5, by enzymatic synthesis with a glycoside phosphorylases (GP) that links monomeric GlcNAc via a β-1,3-glycosidic linkage.

The hydroxyl groups at C4 may be equatorial. The hydroxyl groups at C4 may be axial.

The integer n may be between 10 and 50. The integer n may be between 10 and 100. The integer n may be between 10 and 200. The integer n may be between 10 and 300. The integer n may be between 10 and 400. The integer n may be between 10 and 500. The integer n may be between 10 and 600. The integer n may be between 10 and 700. The integer n may be between 10 and 800. The integer n may be between 10 and 900. The integer n may be between 10 and 1,000. The integer n may be between 10 and 2,000. The integer n may be between 10 and 3,000. The integer n may be between 10 and 4,000. The integer n may be between 10 and 5,000. The integer n may be between 10 and 6,000. The integer n may be between 10 and 7,000. The integer n may be between 10 and 8,000. The integer n may be between 10 and 9,000. The integer n may be between 10 and 10,000.

X may be —OH.

The polysaccharide may be purified. The polysaccharide may be lyophilized.

The polysaccharide may forms part of a pharmaceutical composition, a cosmetic composition, a food composition, or a beverage composition. The polysaccharide may be for use as a part of a pharmaceutical composition, a cosmetic composition, a food composition, a beverage composition, a vaccine composition, or as a coating for a textile or as a coating for a medical device.

The method of claim 11, wherein the GP enzyme may be a β-1,3-GlcNAc phosphorylase.

The method of claim 11 or 12, wherein the glycoside phosphorylase may include an amino acid sequence that may be at least 95% identical to SEQ ID NO: 2 and wherein the enzyme has β-1,3-GlcNAc phosphorylase enzyme activity.

The method of claim 11, 12, or 13, wherein the β-1,3-GlcNAc phosphorylase enzyme may be the phosphorylase peptide including an amino acid sequence that may be identical to SEQ ID NO: 2.

The method may further include: (c) precipitating the oligosaccharide or polysaccharide product from the reaction mixture of step (b); and (d) purifying the oligosaccharide or polysaccharide product from the reaction mixture of step (c).

The method may further include lyophilizing the oligosaccharide or polysaccharide product from the reaction mixture of step (d). Step (a) may be carried out in a first reaction chamber and step (a) may be carried out in a second reaction chamber. Step (a) may be carried out for 18 h at 37° C. and step (b) may be carried out for 48 h at room temperature.

The molar ratio of GlcNAc or GalNAc:ATP may be 1:1.3. The NahK may be isolated from Bifidobacterium longum including an amino acid sequence that may be at least 95% identical to SEQ ID NO: 1 and wherein the enzyme has NahK enzyme activity. The GlcNAc-1-P may be alternatively produced by phosphorolysis of chitin or N,N-di-acetylchitobiose using a chitobiose phosphorylase or a chitinase. The chitobiose phosphorylase may include an amino acid sequence that may be at least 95% identical to SEQ ID NO: 4 or a chitinase may include an amino acid sequence that may be at least 95% identical to SEQ ID NO: 5

The glycosyl acceptor may be GlcNAc. The glycosyl acceptor may be GalNAc.

The donor:acceptor ratio may be at least 100:1. The donor:acceptor ratio may be at least 1000:1.

The method may further include continual removal of the phosphate from the reaction solution. The removal of the phosphate from the reaction solution may be by precipitation. The precipitation may be with a counter ion. The counter ion may be barium acetate. The glycoside phosphorylase may be a β-1,3-glycoside phosphorylase. The glycoside phosphorylase has binding sites specific for GlcNAc-1-P as a glycosyl donor and GlcNAc as a glycosyl acceptor. The glycoside phosphorylase has binding sites specific for GalNAc-1-P as a glycosyl donor and GalNAc as a glycosyl acceptor. The glycoside phosphorylase may be a β-1,3-GlcNAc phosphorylase isolated from the mycobacterium Acholeplasma laidlawii. The β-1,3-GlcNAc phosphorylase enzyme may be a phosphorylase peptide including an amino acid sequence that may be at least 95% identical to SEQ ID NO:2. The NahK enzyme may be a peptide including an amino acid sequence that may be at least 95% identical to SEQ ID NO:1.

The donor:acceptor ratio may be at least 100:1. The donor:acceptor ratio may be at least 1000:1.

The glycoside phosphorylase may be a β-1,3-GlcNAc phosphorylase. The glycoside phosphorylase may be a β-1,3-GlcNAc phosphorylase isolated from the mycobacterium Acholeplasma laidlawii. The β-1,3-GlcNAc phosphorylase enzyme may be the phosphorylase peptide including an amino acid sequence that may be at least 95% identical to SEQ ID NO:2. The β-1,3-GlcNAc phosphorylase enzyme may be the phosphorylase peptide including an amino acid sequence that may be identical to SEQ ID NO:2.

The donor:acceptor ratio may be of at least 50:1. The may donor:acceptor ratio may be of at least 60:1. The donor:acceptor ratio may be of at least 70:1. The may donor:acceptor ratio may be of at least 80:1. The donor:acceptor ratio may be of at least 90:1. The donor:acceptor ratio may be of at least 100:1. The may donor:acceptor ratio may be of at least 110:1. The donor:acceptor ratio may be of at least 120:1. The may donor:acceptor ratio may be of at least 130:1. The donor:acceptor ratio may be of at least 140:1. The may donor:acceptor ratio may be of at least 150:1. The donor:acceptor ratio may be of at least 160:1. The may donor:acceptor ratio may be of at least 170:1. The donor:acceptor ratio may be of at least 180:1. The may donor:acceptor ratio may be of at least 190:1. The donor:acceptor ratio may be of at least 200:1. The may donor:acceptor ratio may be of at least 300:1. The donor:acceptor ratio may be of at least 400:1. The may donor:acceptor ratio may be of at least 500:1. The donor:acceptor ratio may be of at least 600:1. The may donor:acceptor ratio may be of at least 700:1. The donor:acceptor ratio may be of at least 800:1. The may donor:acceptor ratio may be of at least 900:1. The donor:acceptor ratio may be of at least 1,000:1. The may donor:acceptor ratio may be of at least 1,500:1. The donor:acceptor ratio may be of at least 2,000:1. The may donor:acceptor ratio may be of at least 2,500:1. The donor:acceptor ratio may be of at least 3,000:1. The may donor:acceptor ratio may be of at least 3,500:1. The donor:acceptor ratio may be of at least 4,000:1. The may donor:acceptor ratio may be of at least 4,500:1. The donor:acceptor ratio may be of at least 5,000:1. The may donor:acceptor ratio may be of at least 5,500:1. The donor:acceptor ratio may be of at least 6,000:1. The may donor:acceptor ratio may be of at least 6,500:1. The donor:acceptor ratio may be of at least 7,000:1. The may donor:acceptor ratio may be of at least 7,500:1. The donor:acceptor ratio may be of at least 8,000:1. The may donor:acceptor ratio may be of at least 8,500:1. The donor:acceptor ratio may be of at least 9,000:1. The may donor:acceptor ratio may be of at least 9,500:1. The donor:acceptor ratio may be of at least 10,000:1.

The degree of polymerization (DP) may be at least 6. The degree of polymerization (DP) may be at least 7. The DP may be at least about 8. The DP may be at least about 9. The DP may be at least about 10. The DP may be at least about 11. The DP may be at least about 12. The DP may be at least about 13. The DP may be at least about 14. The DP may be at least about 15. The DP may be at least about 16. The DP may be at least about 17. The DP may be at least about 18. The DP may be at least about 19. The DP may be at least about 20. The DP may be at least about 21. The DP may be at least about 22. The DP may be at least about 23. The DP may be at least about 24. The DP may be at least about 25. The DP may be at least about 26. The DP may be at least about 27. The DP may be at least about 28. The DP may be at least about 29. The DP may be at least about 30. The DP may be at least about 40. The DP may be at least about 50. The DP may be at least about 60. The DP may be at least about 70. The DP may be at least about 80. The DP may be at least about 90. The DP may be at least about 100. The DP may be at least about 150. The DP may be at least about 200. The DP may be at least about 300. The DP may be at least about 400. The DP may be at least about 500. The DP may be at least about 600. The DP may be at least about 700. The DP may be at least about 800. The DP may be at least about 900. The DP may be at least about 1,000. The DP may be at least about 1,500. The DP may be at least about 2,000. The DP may be at least about 2,500. The DP may be at least about 3,000. The DP may be at least about 3,500. The DP may be at least about 4,000. The DP may be at least about 4,500. The DP may be at least about 5,000. The DP may be at least about 5500. The DP may be at least about 6,000. The DP may be at least about 6,500. The DP may be at least about 7000. The DP may be at least about 7,500. The DP may be at least about 8,000. The DP may be at least about 8,500. The DP may be at least about 9,000. The DP may be at least about 9,500. The DP may be at least about 10,000.

The acceptor may be an oligosaccharide or a polysaccharide a non-reducing end of a GlcNAc or GalNAc. The acceptor may be a chitin or a chito-oligosaccharide or a chito-polysaccharide. The β-1,3-GlcNAc phosphorylase enzyme may be the phosphorylase peptide including an amino acid sequence that may be at least 95% identical to SEQ ID NO: 2 and wherein the enzyme has β-1,3-GlcNAc phosphorylase enzyme activity. The β-1,3-GlcNAc phosphorylase enzyme may be the phosphorylase peptide including an amino acid sequence that may be identical to SEQ ID NO: 2.

The molar ratio of GlcNAc or GalNAc:ATP may be between 1:1 and 1:1.1. The molar ratio of GlcNAc or GalNAc:ATP may be between 1:1 and 1:1.2. The molar ratio of GlcNAc or GalNAc:ATP may be between 1:1 and 1:1.3. The molar ratio of GlcNAc or GalNAc:ATP may be between 1:1 and 1:1.4. The molar ratio of GlcNAc or GalNAc:ATP may be between 1:1 and 1:1.5. The molar ratio of GlcNAc or GalNAc:ATP may be between 1:1 and 1:1.6. The molar ratio of GlcNAc or GalNAc:ATP may be between 1:1 and 1:1.7. The molar ratio of GlcNAc or GalNAc:ATP may be between 1:1 and 1:1.8. The molar ratio of GlcNAc or GalNAc:ATP may be between 1:1 and 1:1.9. The molar ratio of GlcNAc or GalNAc:ATP may be between 1:1 and 1:2. The molar ratio of GlcNAc or GalNAc:ATP may be between 1:1 and 1:3. The molar ratio of GlcNAc or GalNAc:ATP may be between 1:1 and 1:4. The molar ratio of GlcNAc or GalNAc:ATP may be between 1:1 and 1:5. The molar ratio of GlcNAc or GalNAc:ATP may be between 1:1 and 1:6. The molar ratio of GlcNAc or GalNAc:ATP may be between 1:1 and 1:7. The molar ratio of GlcNAc or GalNAc:ATP may be between 1:1 and 1:8. The molar ratio of GlcNAc or GalNAc:ATP may be between 1:1 and 1:9. The molar ratio of GlcNAc or GalNAc:ATP may be between 1:1 and 1:10. The molar ratio of GlcNAc or GalNAc:ATP may be between 1:1 and 1:15. The molar ratio of GlcNAc or GalNAc:ATP may be between 1:1 and 1:20. The molar ratio of GlcNAc or GalNAc:ATP may be between 1:1 and 1:25. The molar ratio of GlcNAc or GalNAc:ATP may be between 1:1 and 1:30.

The β-1,3-GlcNAc phosphorylase enzyme may be a phosphorylase peptide including an amino acid sequence that may be at least 95% identical to SEQ ID NO:2 and having β-1,3-GlcNAc phosphorylase enzyme activity.

The NahK enzyme may be a peptide including an amino acid sequence that may be at least 95% identical to SEQ ID NO:1 and having NahK enzyme activity.

In one embodiment there is provided oligosaccharides or polysaccharides comprising repeated monomers of N-acetyl-glucosamine (GlcNAc—an amine derivative of glucose having the general formula C₈H₁₅NO₆) linked by glycosidic bonds having a β-configuration between the C1 position of the first GlcNAc ring and the C3 position of the adjacent GlcNAc ring and having the following structure (where n=number of repeated GlcNAc monomers) and for the current example the hydroxyl groups at C4 are equatorial.

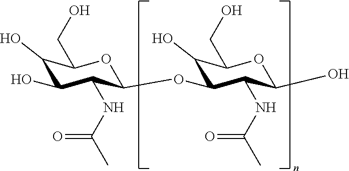

Oligosaccharides as described herein have less than ten (10) monomers of GlcNAc linked by β-1,3-glycosidic bonds. Polysaccharides as described herein have more than ten (10) monomers of GlcNAc linked by β-1,3-glycosidic bonds. Alternatively, polysaccharides as described herein have ten (10) or more monomers of GlcNAc linked by β-1,3-glycosidic bonds.

In another embodiment are oligosaccharides or polysaccharides as described herein synthesized through the use of enzymes, preferably glycoside phosphorylases (GP), that link monomeric GlcNAc via a β-1,3-glycosidic linkage.

In another embodiment are methods for synthesizing oligosaccharides or polysaccharides as described herein comprising the following steps: a) Generation of GlcNAc-1-P, as a glycosyl donor, preferably by reacting an N-acetylhexosamine-1-kinase (NahK) with GlcNAc and ATP (at molar ration of 1:1.3) in a reaction chamber for 18 h at 37° C. N-acetylhexosamine-1-kinases may include but is not limited to NahK isolated from Bifidobacterium longum. GlcNAc-1-P can also be generated by phosphorolysis of chitin or N,N-di-acetylchitobiose using suitable glycoside phosphorylases and, as needed, chitinases. b) In a second reaction chamber, initiation of a reverse phosphorolysis reaction by mixing the GlcNAc-1-P precipitate from step (a) with a glycosyl acceptor, preferably GlcNAc (at a donor:acceptor ratio of at least 1000:1) with a glycoside phosphorylase for 48 h at room temperature. The glycoside phosphorylase described herein is preferably a β-1,3-glycoside phosphorylase with binding sites specific for GlcNAc-1-P as a glycosyl donor and GlcNAc as a glycosyl acceptor and more preferably a β-1,3-GlcNAc phosphorylase isolated from the mycobacterium Acholeplasma laidlawii and yet more preferably the enzyme purified from the E. coli DNA plasmid pET45b containing the DNA sequence that encodes the acholetin phosphorylase peptide sequence (GenBank ID: ABX81671.1 (SEQ ID NO:2)). c) Precipitation, purification and lyophilization of the oligosaccharide or polysaccharide product from the reaction mixture of step (b) by standard methods known in the art.

In another embodiment are oligosaccharides and polysaccharides as described herein for the treatment or diagnosis of medical conditions wherein the activity or physical characteristic of the oligosaccharide or polysaccharide is beneficial; as a component of cosmetic, food or beverage products; as a component for the delivery of pharmaceuticals or as a component of a vaccine; for use as a coating for textile or medical devices.

Alternatively, there may be 90% sequence identity as defined by NCBI BLAST sequence similarity search tool, using the default settings for nucleotide searching or protein searching. Alternatively, there may be 90% sequence similarity as defined by NCBI BLAST sequence similarity search tool, using the default settings for nucleotide searching or protein searching. The sequence similarity may be at least 91%. The sequence similarity may be at least 92%. The sequence similarity may be at least 93%. The sequence similarity may be at least 94%. The sequence similarity may be at least 95%. The sequence similarity may be at least 96%. The sequence similarity may be at least 97%. The sequence similarity may be at least 98%. The sequence similarity may be at least 99%. Alternatively, the sequence identity may be at least 91%. The sequence identity may be at least 92%. The sequence identity may be at least 93%. The sequence identity may be at least 94%. The sequence identity may be at least 95%. The sequence identity may be at least 96%. The sequence identity may be at least 97%. The sequence identity may be at least 98%. The sequence identity may be at least 99%. The sequence similarity or the sequence identity may be determined by NCBI BLAST sequence similarity search tool, using the default settings for nucleotide searching or protein searching.

Furthermore, it will be appreciated by a person of skill in the art, that the enzymes described herein may be expressed in plasmids, integrated into the bacterial chromosome or some combination of both.

The sequences described herein may include whatever promoters, cofactors, ribosomal binding sites etc. as may be required to effectively transcribe, translate and post-translationally modify the enzyme with a high degree of efficiency.

### BRIEF DESCRIPTION OF THE DRAWINGS

FIG. 1 shows a functional characterization of AchP. (A) Purified AchP donor and acceptor specificity screen. AchP donor and acceptor specificity screen. Activity was monitored by coupling phosphate release from reverse phosphorolysis to the formation of molybdenum blue, which absorbs strongly at 655 nm. (B) AchP reaction scheme. (C) AchP degree of polymerization (DP) analysis. DP was characterized using MALDI-MS with donors, Glc1-P, GlcNAc1-P or GalNAc1-P and GalNAc, in either 1:10, 1:1 or 100:1 donor-to-acceptor ratios. m/z peaks correspond to the acholetin+Na adducts [M+Na]. Max: largest acholetin chain detected. DP analysis with GalNAc as acceptor is shown in FIG. 6.

FIG. 2 shows a two-pot large scale acholetin synthesis. (A) Pot one shows NahK catalyzed production of GlcNAc1-P. Sequential barium precipitation was performed to reduce ADP, AMP and inorganic phosphate concentrations following the completion of the reaction. GlcNAc1-P preparation was precipitated with ethanol then dried. (B) Pot two shows AchP catalyzed production of acholetin. Following the completion of the reaction the acholetin sample was desalted with G25 resin (FIG. 9) prior to lyophilization and product analysis. (C) Acholetin HMBC analysis. Overlapping correlations were resolved with the help of ¹H and ¹³C COSY and HSQC experiments (FIG. 11).

FIG. 3: shows E. coli cell lysate AchP donor and acceptor specificity screen. Cell lysate from E. coli expressing AchP was combined with the indicated donor and acceptor combinations. Activity was monitored by coupling phosphate release from reverse phosphorolysis to the formation of molybdenum blue, which absorbs strongly at 655 nm. Grey indicates corresponding donor/acceptor combination not tested.

FIG. 4: shows AchP product analysis TLC. (A) AchP products with GlcNAc1-P (donor) and GlcNAc (GNAc1), GlcNAc-β1,4-GlcNAc (GNAc2), or GlcNAc(-β1,4-GlcNAc)3 (GNAc4) (acceptors) were analyzed by TLC, where the products are outlined in black boxes. (B) AchP forward phosphorolysis product analysis with GlcNAc-β1,4-GlcNAc and phosphate and the reverse phosphorolysis with Glc1-P and GlcNAc was include as a control reaction.

FIG. 5: shows reverse phosphorolysis mechanism of a β-inverting CDP contrasting glucose and galactose as acceptors.

FIG. 6: shows AchP degree of polymerization (DP) analysis. DP was characterized using MALDI-MS with donors, Glc1-P, GlcNAc1-P or GalNAc1-P and acceptor, GalNAc, in either 1:10, 1:1 or 100:1 donor-to-acceptor ratios.

FIG. 7: shows Core 3 mucin-like O-glycan analog synthesis with AchP. (A and B) TLC and MS analysis of AchP catalyzed products with GlcNAc1-P (donor) and either GalNAc-β-MU or Gal-NAc-α1-pNP (acceptor). (C and D) Cleavage of the GlcNAc-β1,3-GalNAc moiety from GlcNAc-Pβ1,3-GalNAc-α1-MU or β1,3-GalNAc-α1-pNP and release of the fluoro-/chromogenic aglycone catalyzed by EngSP.

FIG. 8: shows acholetin enzymatic degradation assay. spHex, AchP, gsChit and dspB were incubated with acholetin (top), chitin (middle) or PNAG (bottom) for 2 h at room temperature, where 50 mM phosphate (pH 7.0) was included in all reactions.

FIG. 9: shows G25 acholetin desalting, (A) UV absorbance (grey) and conductance trace (black). Vertical red lines indicate fraction collections and (B) TLC analysis of acholetin fractions from G25 column.

FIG. 10: shows GlcNAc-β1,3-GlcNAc HMBC showing the β1,3-linkage. Dark grey line: Signals corresponding to the reducing end GlcNAc unit. Light grey line: Signals corresponding to the non-reducing end GlcNAc unit. Dark grey and light grey: 1H-13C correlations between two GlcNAc units.

FIG. 11: shows 13C NMR of untreated acholetin compared to reduced-end reduced acholetin, where the carbon numbers are shaded to correspond to the acholetin structure shown at the top, where the reducing end carbons (numbers) are shaded in to indicate their shift following reduction.

FIG. 12: shows AchP reverse phosphorolysis Michaelis-Menten plots, where the reactions were carried out with 10 mM GlcNAc1-P or Glc1-P donors and varying concentrations of either GlcNAc or GalNAc. Related to TABLE 2.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

Any terms not directly defined herein shall be understood to have the meanings commonly associated with them as understood within the art of the invention.

### GH94 Family Enzymes

The GH94 family contained a potential AchP enzyme identified from a set of 1161 GH94 amino acid sequences with an E-value threshold of 10⁻²⁰⁰, corresponding to a pairwise sequence identity over 40%^{35-38, 62}. Prior to this research, the GH94 family contained six known activities (TABLE 1) that are represented within 31 functionally characterized members. The overlaid functional annotations were based on the list of characterized GH94s in the CAZy DB³⁰ (www.cazy.org), characterized metagenomically derived GH94s reported previously³⁹ and GH94 sequences found in the A. laidlawii genome⁴⁰. AchP was found to be one of 23 singletons that do not share a pairwise sequence identity of over 40% with any other GH94 sequence. The member which shared AchP's lowest E-value of 1.1×10⁻¹⁷⁵, with a pairwise score of 37% resided as a doublet in cluster 94-17. The member that shared the lowest E-value (9.5×10⁻²⁷) which also belonged to a cluster containing functionally characterized members is located in 94-1B. However, the E-value between these two sequences fell well short of the threshold and therefore provided no information toward determining AchP's activity. Given that SSN analysis failed to cluster AchP with any characterized GH94 that may suggest its activity, but instead classified it as a singleton, we considered the possibility that AchP may represent a new activity within the GH94 family.

TABLE 1
GH94 activities reported in the CAZy DB. Note, a cyclic 1,2-β-oligoglucan
phosphorylase is also reported in the CAZy DB, however, this activity is
classified with the same EC# (2.4.1.333) as (non-cyclic) 1,2-β-oligoglucan
phosphorylase, therefore the two are combined here under a single activity.

| | | | Phosphorolysis |
|---|---|---|---|
| EC | Name | Substrate | Product |
| 2.4.1.20 | Cellobiose phosphorylase | Glc-β1,4-Glc | Glc-α1-P |
| 2.4.1.31 | Laminaribiose phosphorylase | Glc-β1,3-Glc | Glc-α1-P |
| 2.4.1.49 | Cellodextrin phosphorylase | Glc-(β1,4-Glc)ᵣ | Glc-α1-P |
| 2.4.1.280 | N,N′-Diacetylchitobiose phosphorylase | GlcNAc-β1,4-GlcNAc | GlcNAc-α1-P |
| 2.4.1.321 | Cellobionic acid phosphorylase | Glc-β1,4-Gluconic acid | Glc-α1-P |
| 2.4.1.333 | 1,2-β-Oligoglucan phosphorylase | Glc-(β1,2-Glc)ᵣ | Glc-α1-P |

### Substrate Specificity Screen

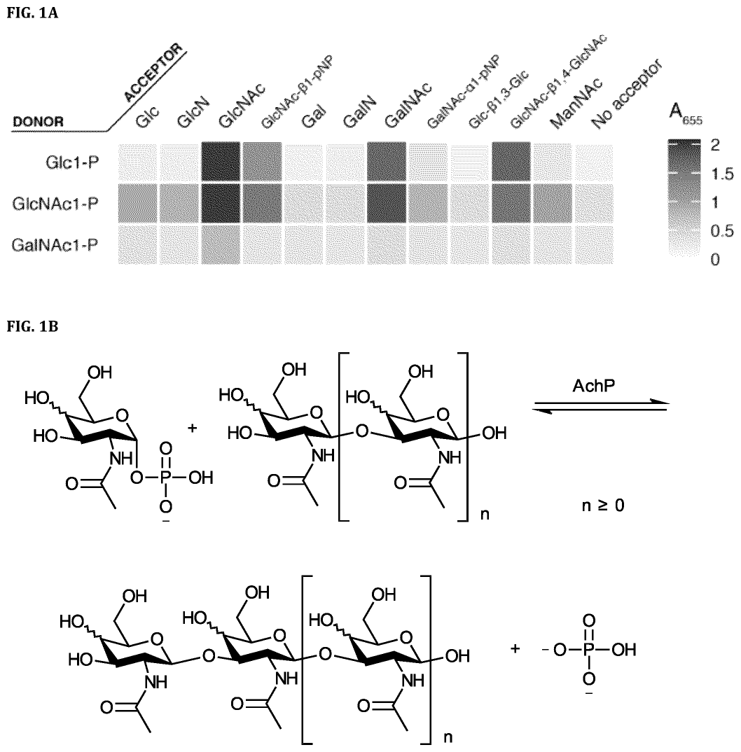

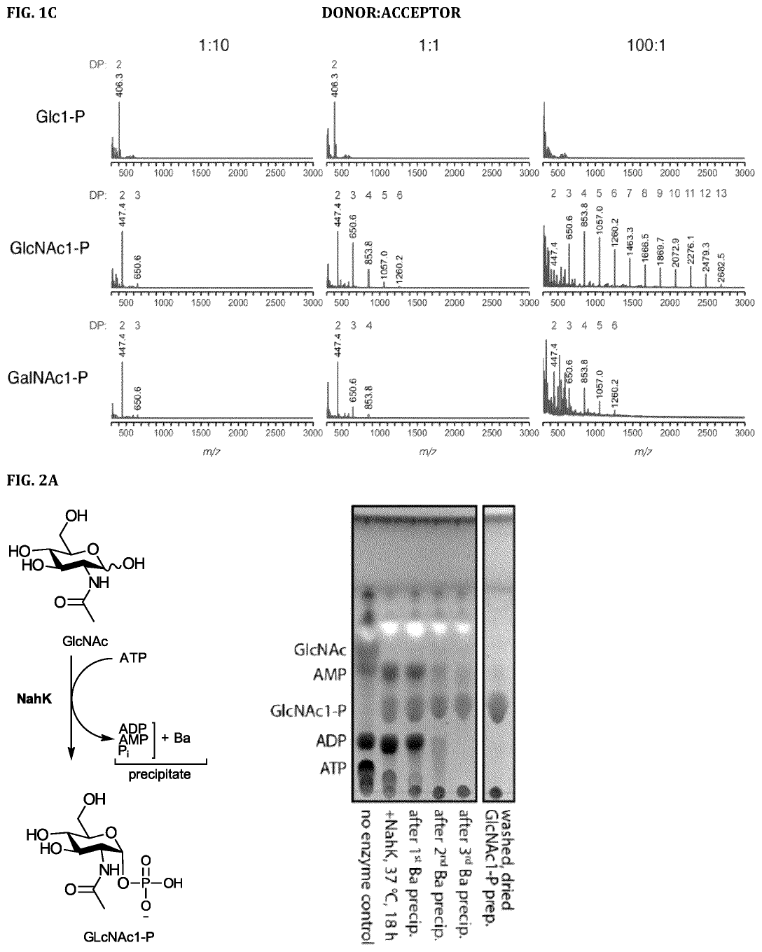

AchP was heterologously expressed, purified and screened, in the absence of cell lysate, against an extended set of donors and acceptors, including additional N-acetamido sugars, using a phosphorylase screening method described previously that couples the chromogenic development of molybdenum blue to the liberation of free phosphate during reverse phosphorolysis^{39,41} (FIG. 1A). When incubated with Glc1-P as donor, AchP was active in the presence of the acceptors GlcNAc, GlcNAc-β1-pNP, GalNAc and GlcNAc-β1,4-GlcNAc, while no activity was detected with the non-acetylated glucosamine and galactosamine. With GlcNAc1-P as donor, AchP was active with all acceptors that contained an N-acetyl moiety, as well as with glucose and glucosamine, but not galactose and galactosamine. Lastly, when GalNAc1-P was used as donor, activity could only be detected when Glc-NAc was the acceptor. These results show that GlcNAc1-P is a superior donor relative to Glc1-P and thus that binding of an acetamide in the −1 (donor) subsite is advantageous. Of the 11 acceptors screened, only GlcNAc was able to stimulate activity in the presence of every donor assayed, indicating that again an equatorial acetamide moiety is preferred on the sugar binding in the +1 subsite. Interestingly however GalNAc, with an axial C4 hydroxyl, also proved to be a good acceptor, as does an axial glycoside thereof.

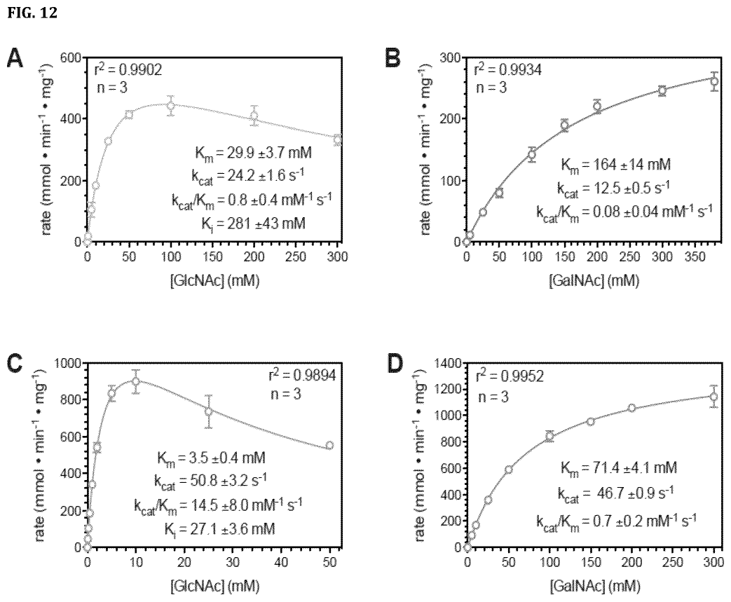

TABLE 2
Reverse phosphorolysis kinetic parameters for AchP. Reactions were carried
out with 10 mM GlcNAc1-P or Glc1-P donors and varying concentrations of
either GlcNAc or GalNAc. Michaelis-Menten plots are shown in FIG. 12.

| | | | Kₘ | k_{cat} | k_{cat}/Kₘ | Kᵢ |
|---|---|---|---|---|---|---|
| FIG. | Donor | Acceptor | (mM) | (s⁻¹) | (mM⁻¹ s⁻¹) | (mM) |
| A | αGlc1-P | GlcNAc | 29.9 ± 3.7 | 24.2 ± 1.6 | 0.8 ± 0.4 | 281 ± 43 |
| B | αGlc1-P | GalNAc | 164 ± 14 | 12.5 ± 0.5 | 0.08 ± 0.04 | N/A |
| C | αGlcNAc1-P | GlcNAc | 3.5 ± 0.4 | 50.8 ± 3.2 | 14.5 ± 8.0 | 27.1 ± 3.6 |
| D | αGlcNAc1-P | GalNAc | 71.4 ± 4.1 | 46.7 ± 0.9 | 0.7 ± 0.2 | N/A |

To quantitate this specificity to some degree, kinetic parameters were determined for reaction with the donors, Glc1-P and GlcNAc1-P, each in the presence of either acceptor, GlcNAc or GalNAc (TABLE 2). Activities were too low with the donor GalNAc1-P or the acceptor glucose when using reasonable substrate and enzyme concentrations, so kinetic parameters were not determined. GlcNAc1-P is the preferred donor over Glc1-P, with a Kₘ value almost 10-fold lower and k_{cat} 2-fold greater when using GlcNAc as acceptor, confirming and quantitating the importance of the equatorial C-2 acetamide. The very low activity with GalNAc1-P shows that an axial hydroxyl at C-4 is not well accommodated at the donor site, though it binds well in the acceptor locus.

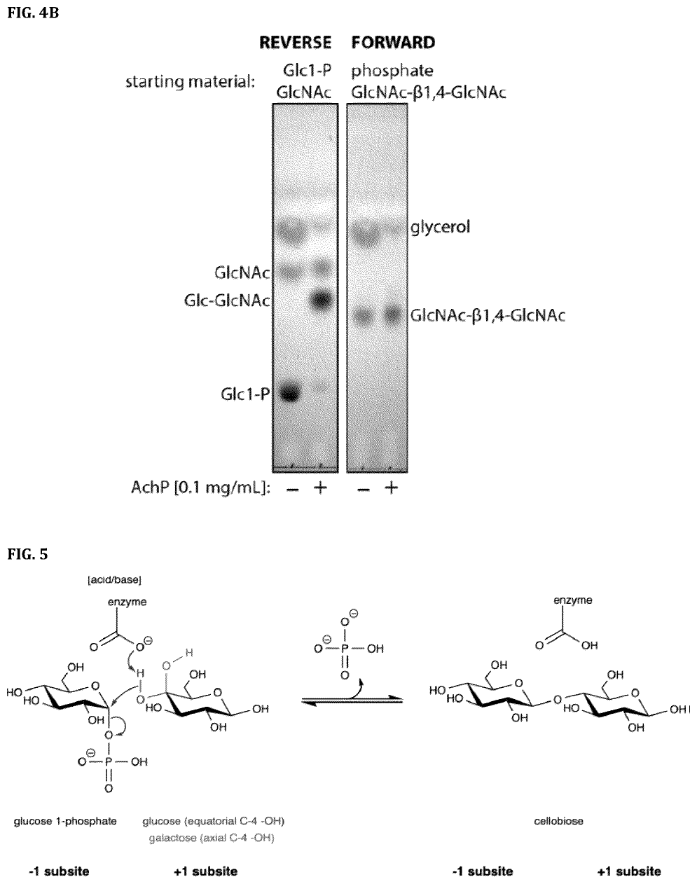

Of the six activities described to date in the GH94 family only the N,N′-diacetylchitobiose phosphorylases (ChbP) are known to use GlcNAc1-P as donor, transferring only to monosaccharide GlcNAc acceptors and not to di- or trisaccharides. The other five types utilize Glc1-P. The bacteria from which both characterized ChbPs were discovered are native to marine environments where chitin, a polysaccharide of β1,4-linked GlcNAc and the primary structural component of the exoskeleton of marine invertebrates, is common. Presumably these ChbPs act on disaccharides released from chitin by chitinases. AchP on the other hand is able to utilize GlcNAc-β1,4-GlcNAc and GlcNAc(-β1,4-GlcNAc)₃ as acceptors performing iterative addition of N-acetylglucosaminyl residues (FIG. 4A). As noted earlier, we initially anticipated that AchP was a chitin phosphorylase. However, when we assayed for phosphorolysis of GlcNAc-β1,4-GlcNAc no substrate cleavage could be detected (FIG. 4B). Although a surprising result at the time, it perhaps should not have been given the previous results showing that AchP transfers to GalNAc, which has an inverted stereochemistry at C-4 relative to GlcNAc. Such transfer would not be expected if the enzyme were 1,4-specific since the hydroxyl at C-4 would be incorrectly oriented (FIG. 5). These results led us to suspect that a different linkage was being formed/broken by AchP.

### Linkage Determination

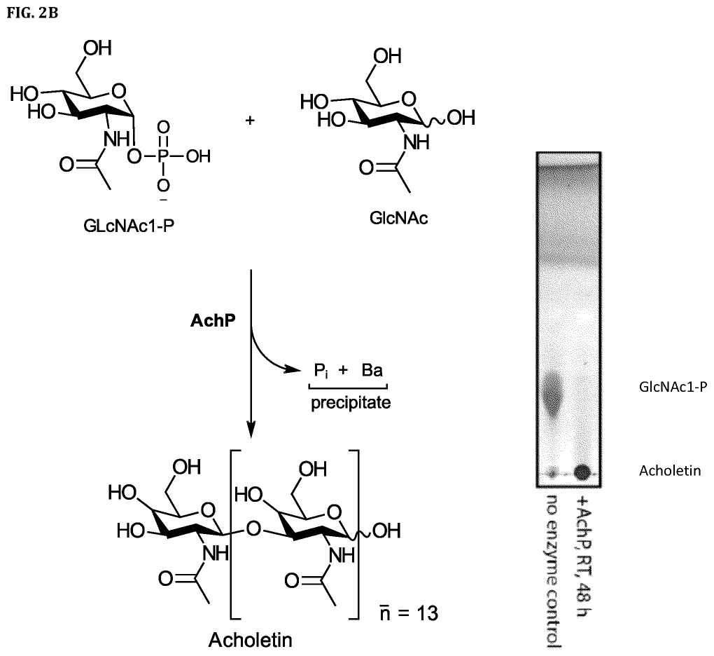

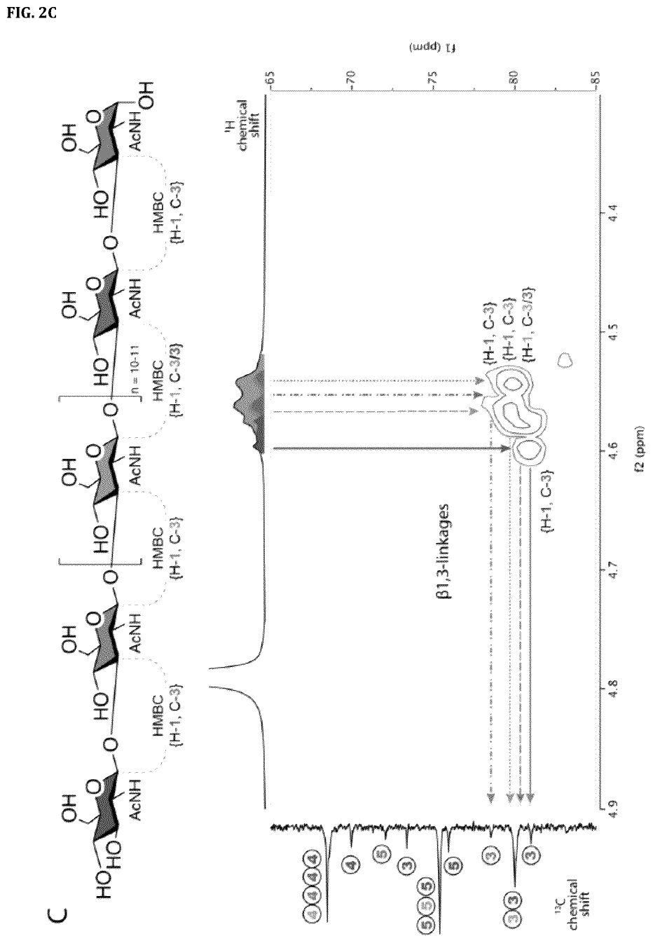

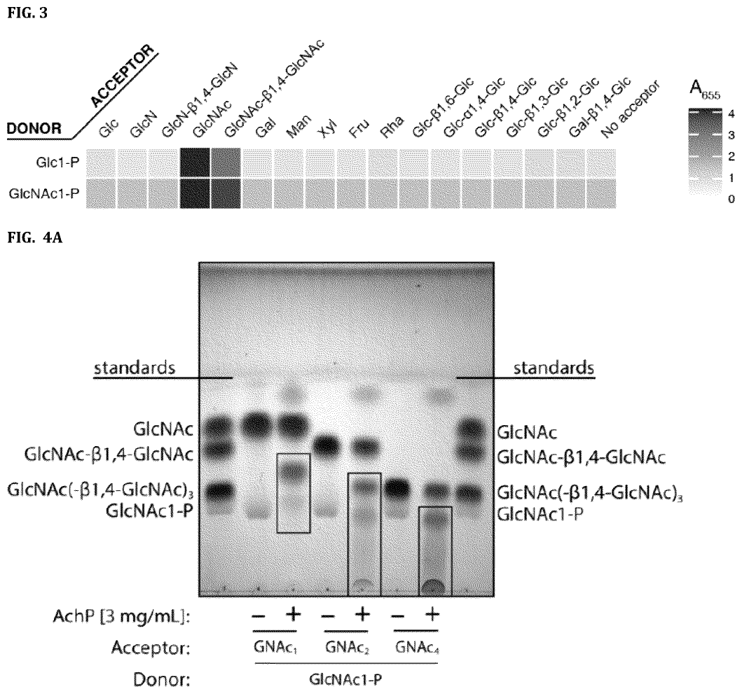

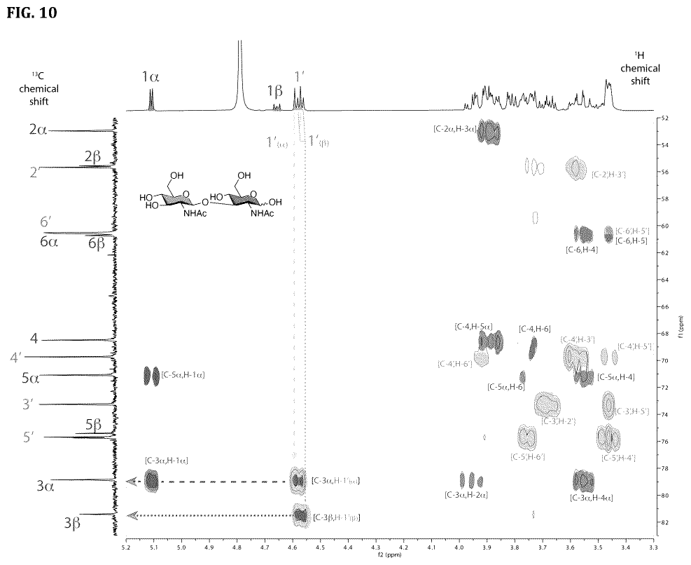

The two other potential linkages AchP could be creating are β1,3 or β1,6, thus product analysis was necessary. Two AchP product glycans were thus analyzed by NMR spectroscopy. The first was the GlcNAc-GlcNAc disaccharide product formed when GlcNAc1-P and GlcNAc were used as the donor/acceptor combination. Strong evidence for a β1,3 glycosidic linkage was obtained from the Heteronuclear Multiple Bond Correlation (HMBC) experiment (FIG. 10). A through-bond correlation was detected between the non-reducing end anomeric pro-ton and the C-3 carbon (α and β, both of which are shifted ˜7 ppm downfield as compared to unlinked GlcNAc) of the reducing end GlcNAc. In order to confirm that use of a different donor does not change the linkage formed we also analyzed the Glc-GlcNAc-α1-pNP produced with Glc1-P as the donor and the aryl glycoside, GlcNAc-α1-pNP as the acceptor. The C-4 and C-6 chemical shifts (68.22, 60.37, respectively) remained approximately equivalent compared to free GlcNAc, whereas the C-3 (82.03) signal was shifted downfield, consistent with the 1,3 linkage. Therefore, through NMR analysis, it was confirmed that AchP generates β1,3 glycosidic linkages when either GlcNAc1-P or Glc1-P were used as donors, as shown in FIG. 2B, when using GlcNAc residues as donor and acceptor.

### Core 3 Mucin-Like O-Glycan Analog

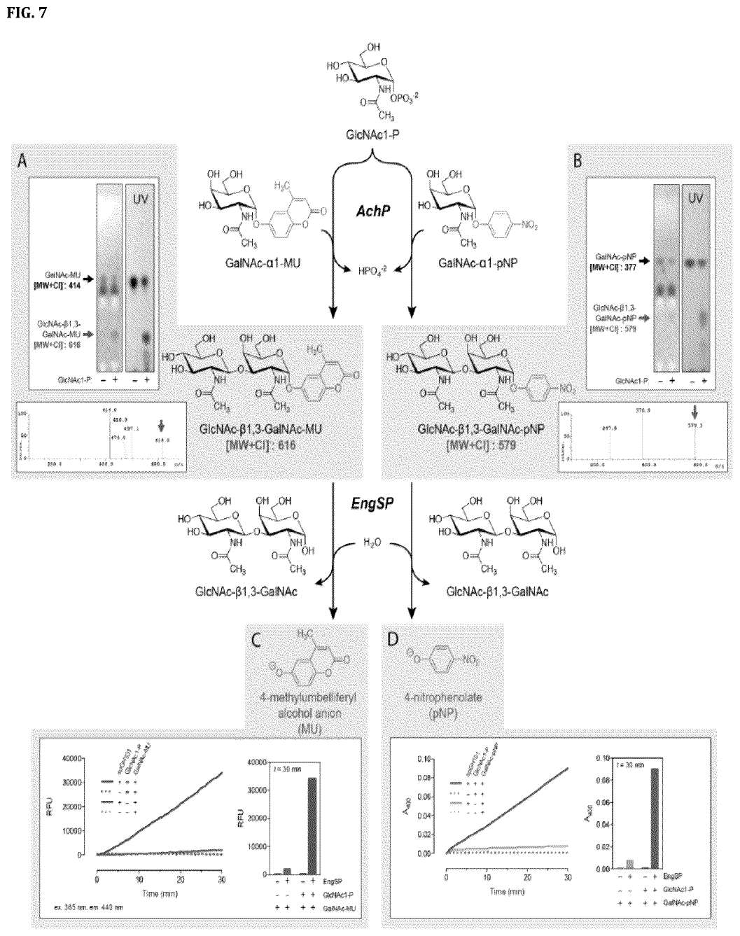

The ability of this enzyme to efficiently transfer GlcNAc to GalNAc with a 1,3 linkage opens the possibility of using this enzyme to make core 3 mucin-type O-glycan structures, GlcNAc-β1,3-GalNAc-α-OR, which can be challenging otherwise due to the instability of the β1,3-GlcNAc transferase responsible for its synthesis in vivo⁴⁶. Using AchP two core 3 analogs were made using GlcNAc1-P as donor and either 4-methylumbelliferyl N-acetyl-α-D-galactosaminide (GalNAc-α1-MU) or GalNAc-α1-pNP as acceptors to generate GlcNAc-β1,3-GalNAc-α1-MU (FIG. 7A) and GlcNAc-β1,3-GalNAc-α1-pNP (FIG. 7B), as confirmed by TLC and mass spectrometry. Further confirmation of product identity was obtained by treatment with a GH101 endo-α-N-acetylgalactosamindase from Streptococcus pneumoniae (EngSP), which is known to cleave the disaccharide moiety from GlcNAc-β1,3-GalNAc-α1-pNP, releasing pNP⁴⁷. After incubation with EngSP, release of both pNP and MU aglycones could be detected (FIGS. 7C and 7D), with only minimal aglycone release in controls.

### Formation of Polymeric Products

In general, glycoside phosphorylases either show a specificity for disaccharides or prefer polymeric substrates, with the GH149 β-1,3-oligoglucan phosphorylases being exceptions in that they display both di- and oligosaccharide phosphorylase activities^{39,44}. AchP appears to also possess both activities since it can use both the monosaccharide, GlcNAc, and disaccharide, N,N′-diacetylchitobiose, as acceptors, with TLC analysis confirming elongation of each acceptor (GlcNAc, GlcNAc-β1,4-GlcNAc and GlcNAc(-β1,4-GlcNAc)3 with the donor, GlcNAc1-P) (FIG. 4A).

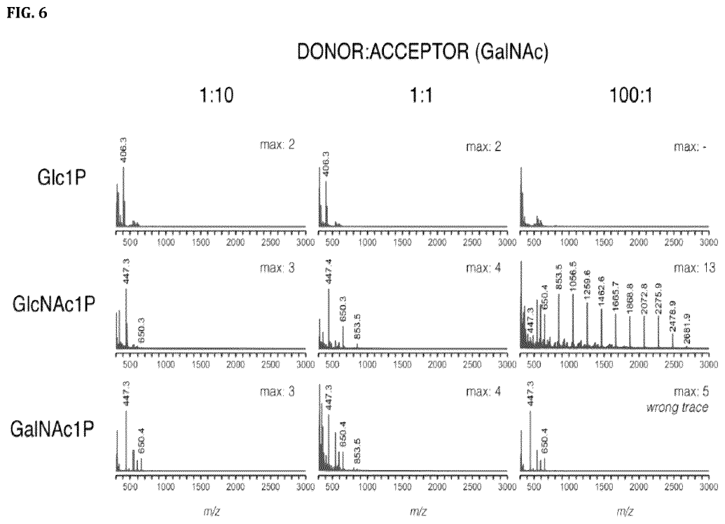

To study the oligomerization further, the degree of polymerization (DP) of the product glycans in the presence of varying concentrations of different donors and acceptors was determined (FIGURE C and FIG. 6). Reactions were carried out in the presence of 10 mM donor and either 100 mM, 10 mM or 0.1 mM GlcNAc, giving donor-to-acceptor ratios of 1:10, 1:1 and 100:1, respectively. When GlcNAc1-P was used as donor the maximum DPs detected by MALDI-MS were 3, 6, and 13 with respective donor-to-acceptor ratios of 1:10, 1:1 and 100:1. When GalNAc1-P was donor the maximum respective DPs were 3, 4, and 6 (FIG. 1C). Similar reactions in which GalNAc replaced GlcNAc as the acceptor (FIG. 6) resulted in the same general pattern of oligomerization. However, when Glc1-P was used at donor-to-acceptor ratios of 1:10 and 1:1, no products with a DP greater than 2 could be detected indicating that the product Glc-β1,3-GlcNAc does not act as an acceptor when Glc1-P is donor. This is consistent with the substrate specificity results described above (FIG. 1A) where no activity could be observed with the donor, Glc1-P, and acceptors, glucose or laminaribiose (Glc-β1,3-Glc), further highlighting the importance of a C-2 acetamide in the +1 subsite. Therefore, like the GH149 β-1,3-oligoglucan phosphorylases, AchP also acts as a dual di- and oligo-saccharide phosphorylase with the donors GlcNAc1-P or Gal-NAc1-P, but when Glc1-P is the donor AchP acts as a strict disaccharide phosphorylase.

### Two-Pot Large Scale Acholetin Synthesis

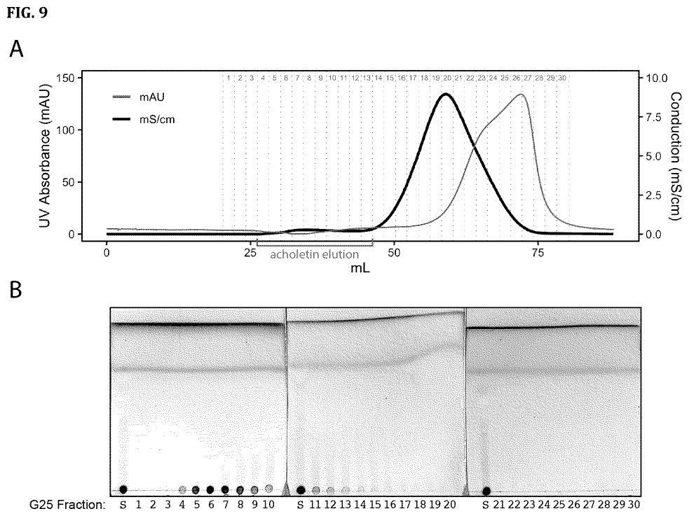

To demonstrate the scalability of the reaction, acholetin synthesis was coupled to GlcNAc1-P production with the aid of an N-acetylhexosamine-1-kinase from Bifidobacterium longum JCM1217 (NahK)⁴⁸ in a two-pot reaction scheme (FIGS. 2A and 2B). In the first pot, GlcNAc was combined with ATP (molar ratio of 1 GlcNAc to 1.3 ATP) and incubated with NahK for 18 h at 37° C. (FIG. 2A). Sequential barium acetate precipitations removed ADP, AMP, free phosphate as well as any unreacted ATP. After the final barium precipitation, GlcNAc1-P, along with some barium acetate, was precipitated with ethanol then washed with acetone before being dried under vacuum. The barium salt was not purified away since it helps by precipitating phosphate liberated during the subsequent AchP reaction, driving the reaction toward synthesis. In the second pot, the GlcNAc1-P preparation was dissolved in buffer containing AchP and GlcNAc at a donor/acceptor ratio of 1000:1 and incubated at room temperature for 48 h, at which point no more GlcNAc1-P could be detected by TLC (FIG. 2B). After removing the precipitated barium phosphate salt and enzyme by heating and centrifugation the reaction mixture was desalted to remove the remaining soluble barium acetate (FIG. 9) then lyophilized to produce a white, fluffy material. The recovered product mass was 344.3 mg, a yield of 37% from the GlcNAc starting material mass of 1 g.

### Acholetin Analysis

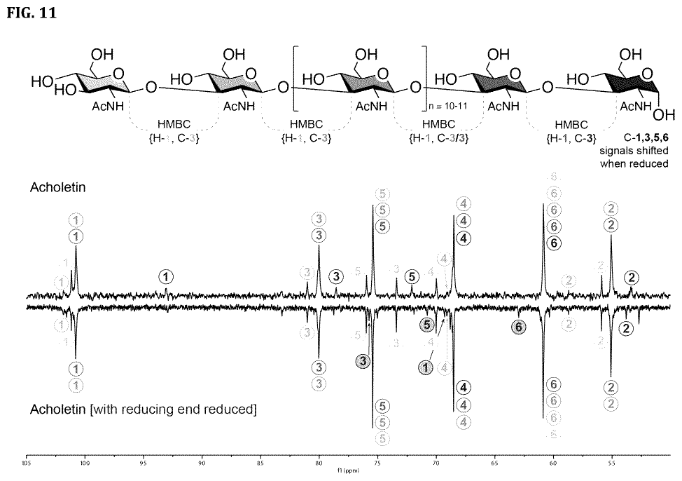

Multi-angle light scattering analysis on the purified acholetin yielded an average molecular weight of 2,966±22 g/mol, indicating an average DP of 14.6 GlcNAc residue per acholetin molecule, while the structure was confirmed by NMR spectroscopy (HMBC). Multiple through-bond correlations corresponding to pairs be-tween H-1 and C-3 from the adjacent unit, established the “HMBC” marked linkages shown on the acholetin structure in FIG. 2C. The presence of both α- and β-anomers at the reducing end and the multiple GlcNAc units made assignment of chemical shifts a challenge. Therefore, the same acholetin sample was reduced with borohydride and the resultant ¹³C NMR spectrum compared with that of the parent sugar to identify resonances from the reducing end unit (which was predominantly α) and correctly assign the corresponding chemical shifts (FIG. 11). Only β-1,3 linkages were found.

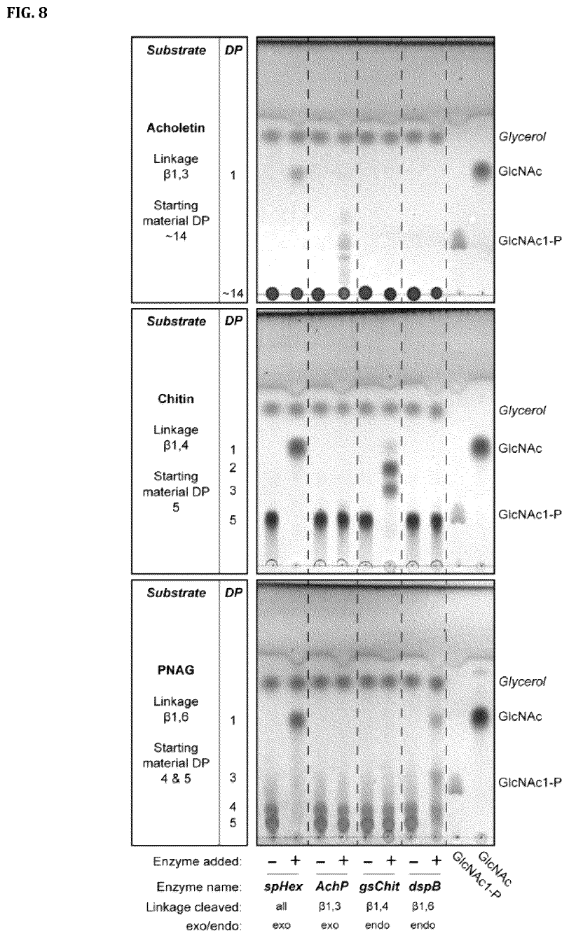

Further confirmation of the linkage type was obtained through enzymatic digestion studies. Treatment of acholetin with either an endochitinase from Streptomyces griseus, which hydrolytically cleaves β1,4 linkages, or with Dispersin B (dspB)⁴⁹, which cleaves β-1,6 linkages resulted in no degradation (FIG. 8). By contrast gsChit and dspB were shown to degrade chito-oligosaccharides and PNAG, respectively, but not acholetin, as would be expected (FIG. 8: middle and bottom). Acholetin was, however, degraded, as also were chitin and PNAG oligos, by an exolytic β-N-acetylhexosaminidase from Streptomyces plicatus (spHex) (FIG. 8: top), which progressively cleaves non-reducing end GlcNAc residues from a range of glycoconjugates, oligosaccharides and polysaccharides⁵⁰⁻⁵².

TABLE 3
Amino Acid Sequences for NahK and AchP

| | SEQ ID | |
|---|---|---|
| Enzyme | NO | Sequence |
| N-acetylhexosamine | 1 | MTESNEVLFGIASHFALEGAVTGIEPYGDGHINTTYLVTTDGPRYILQQMN |
| kinase NahK Enzyme | | TSIFPDTVNLMRNVELVTSTLKAQGKETLDIVPTTSGATWAEIDGGAWRVY |
| from | | KFIEHTVSYNLVPNPDVFREAGSAFGDFQNFLSEFDASQLTETIAHFHDTP |
| Bifidobacterium | | HRFEDFKAALAADKLGRAAACQPEIDFYLSHADQYAVVMDGLRDGSIPLRV |
| longum | | THNDTKLNNILMDATTGKARAIIDLDTIMPGSMLFDFGDSIRFGASTALED |
| (BAF73925.1 or | | EKDLSKVHFSTELFRAYTEGFVGELRGSITAREAELLPFSGNLLTMECGMR |
| JCM1217) | | FLADYLEGDIYFATKYPEHNLVRTRTQIKLVQEMEQKASETRAIVADIMEA |
| | | AR |
| ß-1, 3-GlcNAc | 2 | MKLKQDVISIYQKISLFESGQLNITKLASGAYYLDDELTLITDPVNSGARF |
| phosphorylase | | PYAVNGMTIWAYASGYISINHSSYYILPPNLEGKEPFLDFFGIEQDGNNTY |
| isolated from the | | PVSLLGVSERNDEIENKRYTVFSKNIAYYITVTKNFLYAVTVYISKDEKIY |
| mycobacterium | | FNTVAHNLTGETKQITLSSFFNMLFKYDSGESIETKWFKKVSYENNMFIYD |
| Acholeplasma | | APEDIDRHTRIENYGVVKRHLHTKPKNIQNTTSRIDYVGKRYRSVRNALSI |
| laidlawii | | RSLKFEKAPLVTNFTDTAINADLINYEVKAYDTIISSYRIETCHDKDTLNK |
| GenBank ID: | | MMASDLTDKEIKKVYEGLSNTQSYDFDNFGISFKGVNDNRVDDKVLNQFLK |
| ABX81671 | | LVNYQIHFSSLSSNSGTVFLGVRDVMQQLESSLIWDRKNVRSKILEVLSFI |
| | | DPSGLPPRQYALPPKEGNPRMDLRPFIDQGLWIISTLHTYLAYTEDYDILN |
| | | EVCGYYERIEPNSAKKSKVENSVLEHLIRVTNYLVSNIDPSTYGLKALYGD |
| | | WNDALDGLGLIEGSSGYGNGVSVMATLQLYENLERMIEILKLVDPQNEHIN |
| | | TYEVVRHNLSLGINKYAVVIKQDEKRVLHGWGHDRSYFVGSFNDPDGHSRN |
| | | SLTSNAFYIISDMIKNTPEMKPHLLHAFHNLDSKYGLKTFDPAMQDFHGFG |
| | | RIINLPPGTAENAATYVHATLFGVLALYMLGEGDFANEQVLKVLPITKKEM |
| | | STSPFIMPNSYVHNEELNMDGESMSDWYTGSANTLLKTLIRGLFGLEVKED |
| | | HLRLRPSKAFFSKEATLMVSIGNKLTRIVYKNNNNGNRTFKLNGKVIEAKL |
| | | DTLSGLLYIDINKSILEHQNVIHIQD |

TABLE 4
Nucleic Acid Sequence for AchP

| | SEQ ID | |
|---|---|---|
| Enzyme | NO | Sequence |
| ß-1, 3-GlcNAc | 3 | atgaaactta aacaagatgt aatcagtatt tatcaaaaaa |
| phosphorylase | | tttcactttt tgaaagtggc caattaaata ttacaaagct |
| Acholeplasma | | tgctagtggt gcttactact tagatgatga attaacactt |
| laidlawii strain | | ataacagatc ctgtcaatag tggagctagg tttccatatg |
| DSM 23060 | | ccgtaaatgg tatgaccatt tgggcatatg catcaggtta |
| Ga0215697_11, | | tatttcaata aaccattcat cttactatat actaccgcca |
| whole genome | | aatttagaag gtaaagaacc atttttagat ttttttggaa |
| shotgun | | ttgaacaaga tggtaacaac acatatcctg tgtctttact |
| sequence | | aggtgtatca gagcgtaatg atgaaataga aaataaacgt |
| GenBank ID: | | tacacagtgt ttagtaaaaa tatagcttac tatattacag |
| QRDS01000001 | | ttactaagaa cttcttatat gcggttacag tctatattag |
| 2529 bp DNA linear | | taaagacttt aaaatttact ttaatacagt agcacacaac |
| | | ctaacaggag agaccaaaca aattacactt tcatcattct |
| | | ttaatatgct atttaaatat gatagcggtg aaagtattga |
| | | aacaaaatgg tttaaaaagg taagttatga aaataacatg |
| | | tttatatatg atgcaccaga agatattgat agacacacca |
| | | gaattgaaaa ttatggtgtg gttaaaagac atcttcatac |
| | | aaaacctaaa aacattcaaa atacaacttc aagaattgat |
| | | tatgtaggta aacgttatag atcagttcgt aatgctttaa |
| | | gcattcgaag tctaaaattt gaaaaagcac cccttgttac |
| | | aaattttaca gatactgcaa ttaatgctga tttaattaac |
| | | tacgaagtga aagcctatga taccatcatt agtagttacc |
| | | gtattgaaac atgccacgat aaagatacat taaataagat |
| | | gatggcatct gatttaactg ataaagaaat taaaaaagta |
| | | tatgaaggtt tatctaatac tcaaagttat gattttgata |
| | | actttggcat ctcctttaaa ggtgtaaatg acaatcgagt |
| | | agatgataaa gtcttaaatc agtttttaaa gttagttaat |
| | | taccaaatac acttctcatc cctatcttca aactcaggta |
| | | ccgtattttt aggtgttaga gatgttatgc aacaattaga |
| | | atcatcactg atttgggata gaaaaaatgt aagaagtaaa |
| | | atattagaag tcctttcctt tattgatcca tccggattac |
| | | cgcctagaca atatgcgctt cctcctaaag agggtaatcc |
| | | gaggatggat ttaagaccat ttattgacca aggtctatgg |
| | | attatttcaa cccttcatac ctatttagca tatactgagg |
| | | attatgatat cttaaatgaa gtatgtgggt attatgagcg |
| | | tattgaacca aacagtgcta aaaaatctaa agtagaaaac |
| | | tctgtattag aacacttaat tagagttact aattacttag |
| | | tgtctaatat tgatccaagt acatatggcc taaaggcatt |
| | | atacggagac tggaatgatg cacttgatgg cttaggttta |
| | | attgaaggta gttcaggtta tggtaatggt gtttctgtta |
| | | tggcaacact ccaactatat gaaaacttag aacgcatgat |
| | | tgaaatctta aaactagttg atcctcaaaa tgaacatatt |
| | | aacacatatg aggtggtaag acataattta tctttaggca |
| | | ttaataaata cgcagtcgta ataaagcaag atgaaaaacg |
| | | cgttttacat ggttggggac atgacagaag ttactttgta |
| | | ggtagcttta atgacccgga tggtcactca agaaatagct |
| | | taacctctaa tgctttttat atcatctcag atatgattaa |
| | | aaatacacct gagatgaaac cacatttatt acacgcattc |
| | | cataatcttg actctaaata tggtctaaaa acatttgatc |
| | | cagccatgca agacttccac ggatttggtc gtattataaa |
| | | cctaccacct ggtacagctg aaaatgcagc aacttatgta |
| | | catgcaacac tttttggtgt cttagcactt tatatgttag |
| | | gtgaaggaga ttttgctaat gagcaagtct tgaaagtttt |
| | | acctattact aaaaaggaaa tgtctacatc accatttatt |
| | | atgccaaact catatgtaca caatgaggag cttaacatgg |
| | | acggtgagtc catgagtgac tggtatacag gttctgcaaa |
| | | tacattactt aaaactttaa ttcgtggttt atttggcctt |
| | | gaagtaaagt ttgatcacct aagacttcgt ccatcaaaag |
| | | cattcttctc aaaagaagca acactgatgg taagtattgg |
| | | taacaaactt acacgcatcg tgtataaaaa caataacaac |
| | | ggtaatagaa catttaaatt aaatggtaaa gtaatcgaag |
| | | ctaaactaga tactttaagt ggtctactat atatagatat |
| | | taataaatcc atattggaac accaaaatgt aatacatata |
| | | caagactaa |

TABLE 5
Amino Acid Sequences for ChBP and Chitinase

| | SEQ ID | |
|---|---|---|
| Enzyme | NO | Sequence |
| Chitobiose | 4 | MKYGYFDNDNREYVITRPDVPAPWTNYLGTEKFCTVISHNAGGYSFYNSPE |
| phosphorylase | | YNRVTKFRPNATFDRPGHYVYLRDDDSGDYWSISWQPVAKSLDEAQYQIRH |
| (ChBP) | | GLSYSKFQCDYNGIHARKTLFVPKGEDAEIWDVVIKNTSDQVRTISAFSFV |
| BAC87867.1 | | EFSFSHIQSDNQNHQMSLYSAGTAYRPGLIEYDLYYNTDDFEGFYYLASTF |
| | | DPDSYDGQRDRFLGLYRDEANPLAVEQGRCSNSAQTCYNHCGSLHKQFTLQ |
| | | PGEEIRFAYILGIGKGNGERLREHYQDVANIDAAFAAIKAHWDERCAKFQV |
| | | KSPNQGLDTMINAWTLYQAETCVVWSRFASFIEVGGRTGLGYRDTAQDAIS |
| | | VPHANPEMTRKRIVDLLRGQVKAGYGLHLEDPDWFDPEKEDVAPSKSPTVV |
| | | PTPSDEDKIHGIKDTCSDDHLWLIPTICKYVMETGETSFFDQMIPYADGGE |
| | | ASVYEHMKAALDFSAEYVGQTGICKGLRADWNDCLNLGGGESSMVSFLHFW |
| | | ALQEFIDLAKFLGKDQDVNTYTEMAANVREACETHLWDDEGGWYIRGLTKN |
| | | GDKIGTAQQQEGRVHLESNTLAVLSGLASQERGEQAMDAVDEHLFSPYGLH |
| | | LNAPSFSTPNDDIGFVTRVYQGVKENGAIFSHPNPWAWVAETKLGRGDRAM |
| | | KFYDALNPYNQNDIIEKRIAEPYSYVQFIMGRDHQDHGRANHPWLTGTSGW |
| | | AYFAVTNYILGVQSGFTGLSVDPCIPSDWPGFEVTRQWRGATYHIQVENPD |
| | | HVSKGVKSITLNGAPIQGRIPPQAQGSDNQVVVVLG |
| Chitinase (from | 5 | ATCATAWSSSSVYTNGGTVSYNGRNYTAKWWTQNERPGTSDVWADKGACGT |
| Streptomyces griseus) | | GGEGPGGNNGFVVSEAQFNQMFPNRNAFYTYKGLTDALSAYPAFAKTGSDE |
| 1WVV_A and 1WVU_A | | VKKREAAAFLANVSHETGGLFYIKEVNEANYPHYCDTTQSYGCPAGQAAYY |
| | | GRGPIQLSWNFNYKAAGDALGINLLANPYLVEQDPAVAWKTGLWYWNSQNG |
| | | PGTMTPHNAIVNNAGFGETIRSINGALECNGGNPAQVQSRINKFTQFTQIL |
| | | GTTTGPNLSC |

As used herein “N-acetyl-glucosamine” (GlcNAc) refers to a monosaccharide having the structure:

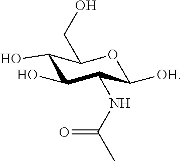

As used herein “N-acetylgalactosamine” (GalNAc) refers to a monosaccharide having the structure:

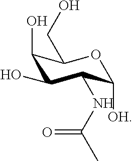

As used herein “N-acetylhexosamine kinase” (NahK) refers to an enzyme having N-acetylhexosamine kinase or N-acetylhexosamine-1-kinase activity. NahK as described herein is useful for the generation of GlcNAc-1-P, as a glycosyl donor, preferably by reacting an NahK with GlcNAc and ATP (at molar ration of 1:1.3). N-acetylhexosamine-1-kinases may include but are not limited to NahK isolated from Bifidobacterium longum. GlcNAc-1-P can also be generated by phosphorolysis of chitin or N,N-di-acetylchitobiose using suitable glycoside phosphorylases and, as needed, chitinases. GlcNAc1-P production with the aid of an N-acetylhexosamine-1-kinase from Bifidobacterium longum JCM1217 (NahK). For example, NahK may have an enzyme having Enzyme protein_id=BAF73925.1 (SEQ ID NO:1). Alternative, NahK sequences may be found at, but not limited to: KAB7788897.1; RSX46818.1; PLS25353.1; PAU69591.1; ALE11460.1; ALE08342.1; KFJ08214.1; KFJ01199.1; KFI92818.1; KFI88272.1; KFI80918.1; KFI56782.1; KFI88848.1; AFL04570.1; 4WH3_A; 4WH2_A; 4WH1_A; WP_250245830.1; WP_250242519.1; WP_250235908.1; WP_250230631.1; WP_237945824.1; WP_230252080.1; WP_225724265.1; WP_217738419.1; WP_212103815.1; WP_211119227.1; WP_204385537.1; WP_197308687.1; WP_196034596.1; WP_195549496.1; WP_195392319.1; WP_015439185.1; WP_193641676.1; WP_191137656.1; WP_174774071.1; WP_174772900.1; WP_161519182.1; WP_154536193.1; WP_154049916.1; WP_144099049.1; WP_143725011.1; WP_143723078.1; WP_136500836.1; WP_131314344.1; WP_131299728.1; WP_131277168.1; WP_131236102.1; WP_131226617.1; WP_131223641.1; WP_131219014.1; WP_131210666.1; WP_131209491.1; WP_131207753.1; WP_131207264.1; WP_131205639.1; WP_131203739.1; WP_131203289.1; WP_117760829.1; WP_115785790.1; WP_115784620.1; WP_114555184.1; WP_106652030.1; WP_106628458.1; WP_106621907.1; WP_101011307.1; WP_101010306.1; WP_077425897.1; WP_077384811.1; WP_077381952.1; WP_077320699.1; WP_071478081.1; WP_065473386.1; WP_065465298.1; WP_065454028.1; WP_065436187.1; WP_052828199.1; WP_052787883.1; WP_032745457.1; WP_032741532.1; WP_032682742.1; WP_025300014.1; WP_025222093.1; WP_021975488.1; WP_019727331.1; WP_014484233.1; WP_013582917.1; WP_012578435.1; WP_011068766.1; WP_010081655.1; WP_008782948.1; WP_007055324.1; WP_007053831.1; WP_003832922.1; WP_003829953.1; DAF63884.1; QUT89213.1; QUT33019.1; QUT27457.1; QUT87333.1; QUT59605.1; QEW38144.1; TSE54269.1; TSE50013.1; RIB34297.1; KWR58128.1; OQC64687.1; ALJ59755.1; and E8MF12.1.

As used herein “β-1,3-GlcNAc phosphorylase” refers to an enzyme having glycoside phosphorylase activity and is also referred to herein as acholetin phosphorylase (AchP). The enzyme is preferably a β-1,3-glycoside phosphorylase with binding sites specific for GlcNAc-1-P as a glycosyl donor and GlcNAc as a glycosyl acceptor. The enzyme may be a β-1,3-GlcNAc phosphorylase isolated from the mycobacterium Acholeplasma laidlawii having the amino acid sequence (GenBank ID: ABX81671.1 (SEQ ID NO:2)).

As used herein “chitinases” are hydrolytic enzymes that break down glycosidic bonds in chitin and may include, but are not limited to chitodextrinase, 1,4-beta-poly-N-acetylglucosaminidase, poly-beta-glucosaminidase, beta-1,4-poly-N-acetyl glucosamidinase, poly[1,4-(N-acetyl-beta-D-glucosaminide)]glycanohydrolase, (1->4)-2-acetamido-2-deoxy-beta-D-glucan glycanohydrolase. Chitinases are generally found in organisms that either need to reshape their own chitin or dissolve and digest the chitin of fungi or animals.

Chito-oligosaccharides (COS) as used herein are the degraded products of chitosan or chitin prepared by enzymatic or chemical hydrolysis of chitosan. The generic structure for COS is shown below, where n=0−8 and R═H or Acetyl group (Ac).

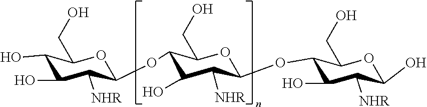

Chitin is the second most abundant naturally occurring polymer after cellulose. Chitin is most commonly found in arthropods (insects, crustaceans, arachnids, and myriapods), nematodes, algae, and fungi. Chitin is a linear polysaccharide composed of (1→4) linked 2-acetamido-2-deoxy-β-d-glucopyranosyl units and occurs naturally in three polymorphic forms with different orientations of the microfibrils, known as α-, β-, and γ-chitin. The α-form has antiparallel chains and is a common and the most stable polymorphic form of chitin found in nature. The β-form of chitin is rare; it occurs in pens of mollusks and is characterized by a loose-packing parallel chains fashion with weak intermolecular interactions and higher solubility and swelling than α-form. The γ-form is characterized by a mixture of antiparallel and parallel chains and was found in the cocoons of insects. Chitin is produced by many living organisms and is usually part of a complex with other polysaccharides and proteins. The structure of chitin is shown below.

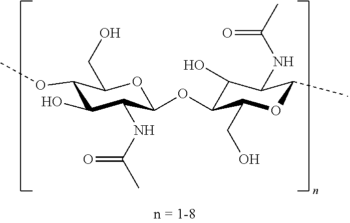

Chitosan is a linear polysaccharide composed of randomly distributed β-(1→4)-linked D-glucosamine (deacetylated unit) and N-acetyl-D-glucosamine (acetylated unit), see below (n=0−8).

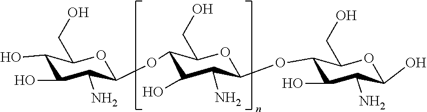

N,N′-Diacetylchitobiose is a dimer of β(1,4) linked N-acetyl-D glucosamine. N,N′-Diacetylchitobiose is the hydrolysate of chitin.

The terms “sequence identity”, “identity” as used herein with respect to polypeptide sequences refer amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the results by 100 to yield the percentage of sequence identity.

Percent identity can be readily determined by any known method, including but not limited to methods known in the art⁶⁷⁻⁷¹. Preferred methods for determining percent identity are designed to give the best match between the sequences tested. Methods of determining identity and similarity are codified in publicly available computer programs, for example. Sequence alignments and percent identity calculations can be performed using the MEGALIGN™ program of the LASERGENE™ bioinformatics computing suite (DNASTAR Inc.™, Madison, Wis.), for example. Multiple alignment of sequences can be performed, for example, using the Clustal™ method of alignment which encompasses several varieties of the algorithm including the Clustal V™ method of alignment 7²,73 and found in the MEGALIGN v8.0 program of the LASERGENE bioinformatics computing suite (DNASTAR Inc.™) For multiple alignments, the default values can correspond to GAP PENALTY=10 and GAP LENGTH PENALTY=10. Default parameters for pairwise alignments and calculation of percent identity of protein sequences using the Clustal™ method can be KTUPLE=1, GAP PENALTY=3, WINDOW=5 and DIAGONALS SAVED=5. Additionally, the Clustal W™ method of alignment can be used⁷²⁻⁷⁴ and found in the MEGALIGN™ v8.0 program of the LASERGENE™ bioinformatics computing suite (DNASTAR Inc.™). Default parameters for multiple alignment (protein/nucleic acid) can be: GAP PENALTY=10/15, GAP LENGTH PENALTY=0.2/6.66, Delay Divergen Seqs (%)=30/30, DNA Transition Weight=0.5, Protein Weight Matrix=Gonnet Series, DNA Weight Matrix=IUB.

Various polypeptide amino acid sequences are disclosed herein as features of certain embodiments. Variants of these sequences that are at least about 70-85%, 85-90%, or 90%-95% identical to the sequences disclosed herein can be used or referenced. Alternatively, a variant amino acid sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identity with a sequence disclosed herein. The variant amino acid sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence. Any polypeptide amino acid sequence disclosed herein not beginning with a methionine can typically further comprise at least a start-methionine at the N-terminus of the amino acid sequence. In contrast, any polypeptide amino acid sequence disclosed herein beginning with a methionine can optionally lack such a methionine residue.

Various alternative embodiments and examples are described herein. These embodiments and examples are illustrative and should not be construed as limiting the scope of the invention.

### MATERIALS AND METHODS

### General Methods

Enzyme quantification was performed using the Bradford method 77. Thin-layer chromatography (TLC) assays were performed on silica gel 60 F254 TLC plates (EMD Millipore Corporation™, Billerica, MA, USA) in a mobile-phase of BuOH:MeOH:NH₄OH:H₂O 5:4:4:1 unless otherwise stated and stained with molybdate TLC stain (2.5% ammonium molybdate (w/v), 1% ceric ammonium sulfate (w/v) and 10% H₂SO₄ (v/v)) or p-anisaldehyde TLC stain (92.5% ethanol, 4% H₂SO₄ (v/v), 1.5% acetic acid (v/v), 2% p-anisaldehyde (v/v)). Visualization of TLC plates was done by heating until the product spots became visible.

### Protein Expression and Purification

### AchP (ABX81671.1 (SEQ ID NO:2))

The DNA encoding the AchP gene was synthesized and inserted into the pET45b expression plasmid by the US Department of Energy Joint Genome Institute. 2 L of LB media containing 100 μg/mL carbenicillin was inoculated with 20 mL of overnight culture of E. coli BL21(DE3) harboring the pET45-h6.AchP plasmid. The culture was grown for 3 h at 37° C., IPTG was added to a final concentration of 0.5 mM, the temperature was reduced to 30° C. and the culture was grown for a further 18 h. Cells were harvested by centrifugation at 6000×g for 10 min in a Beckman Coulter Avanti™ J-E floor centrifuge (JA-10 rotor) followed by resuspension in 40 mL loading buffer (50 mM HEPES pH 7.0, 300 mM NaCl, 10% v/v glycerol, 50 mM MgSO4, 0.1 DTT, 5 mM imidazole). Cells were lysed using an Avestin C3™ homogenizer with an average cell pressure of 16,000 psi. The soluble cell lysate fraction was isolated by centrifuging the crude cell lysate at 15,000 rpm for 30 min (JA-20 rotor). AchP purification was carried out by immobilized metal affinity chromatography on a GH Healthcare AKTA FPLC™ equipped with a UV and conductance detector, an automatic fraction collector and two inlet pumps. Pump A was equilibrated with loading buffer pump B with elution buffer (50 mM HEPES pH 7.0, 300 mM NaCl, 10% v/v glycerol, 50 mM MgSO4, 0.1 mM DTT, 250 mM imidazole). A 5-mL HisTrap™ FF column (GE Healthcare™) were equilibrated with 10 column volumes (CV) of loading buffer. The soluble cell lysate was applied to the column using a P-1 peristaltic pump (GE Healthcare™) followed by a wash step of 10 CV loading buffer. The HisTrap™ column was transferred to the AKTA and washed with 10 CV of 8.2% pump B (25 mM imidazole). AchP was eluted using a 4 CV gradient (8.2-100%) of loading buffer to elution buffer with the fraction collector set to collect 1 mL fractions. Fractions were analyzed by SDS PAGE and the fractions containing the largest bands at 98 kDa were combined and concentrated using Amicon™ Ultra-4 MWCO 30-kDa centrifugal filter (Sigma™). The concentrated protein was diluted with storage buffer (50 mM HEPES pH 7.0, 300 mM NaCl, 10% v/v glycerol, 50 mM MgSO4, 0.1 DTT) in multiple cycles until the imidazole concentration was approximately 1 mM. Final AchP concentration was 12 mg/mL (48 mg total yield) and stored at −70° C.

### NahK

NahK^{63,64} was expressed and purified as described above for AchP, with the following modifications. Expression culture was 3 L. Fraction concentration was done with a 10-kDa centrifugal filter. Final NahK concentration was 6 mg/mL (12 mg total yield).

### AchP (for Crystallography)

The gene was expressed from pET45b using E. coli BL21 (DE3) cells in Terrific Broth media with 0.5 mM IPTG used for induction. The seleno-methionine protein was expressed using the same E. coli strain in PASM-5052 media⁶⁵. Expressions were at 0.5 L scale in 2 L baffled flasks for 2 days at 20° C. and 180 rpm. At harvest the cells were pelleted and stored at −80° C. until thawed for protein purification. The protein was purified using the same protocol whether it was native or seleno-methionine. Cell pellets were removed from the −80° C. freezer and resuspended by stirring with a stir bar in 25 mM HEPES, pH 7.4, 100 mM NaCl, 1 mM DTT (base buffer)+0.1 mg/ml lysozyme, 3 μg/ml DNaseI, 1× Novagen™ EDTA free protease inhibitor. The cells were lysed by multiple passes in the Emulsiflex C3™ emulsifier. The lysate was clarified by centrifugation at 40,000×g for 40 min. The clarified lysate had imidazole added to 30 mM prior to loading onto a 5 mL Histrap™ column on the AKTA purifier HPLC instrument. The HisTrap™ column was equilibrated with base buffer prior to loading of the lysate. The bound protein was eluted with a 0-45% B gradient in 20 CV. Buffer B was the same as base buffer with the addition of 0.5 M imidazole. The elution peak was analyzed by SDS-PAGE and the cleanest fractions were pooled prior to dialysis against 1 L of 25 mM HEPES, pH 7.4, 25 mM NaCl, 1 mM DTT for 2×1 hour at 4° C. At the end of dialysis, the protein showed no precipitation and was loaded onto a 5 mL Q ion exchange column on the FPLC. The bound protein was eluted with a 0-45% B gradient in 24CV. Buffer B was the same as the dialysis buffer but NaCl was 1 M. The elution fractions were analyzed by SDS-PAGE and the cleanest fractions pooled, concentrated and injected onto a 10×300 Superdex™ size exclusion column (SEC). The column was pre-equilibrated with base buffer prior to injecting the protein. The SEC column was run at 0.5 mL/min and the injection volumes were ˜1 mL. The cleanest fractions, as determined by SDS-PAGE analysis, were pooled. The protein was concentrated with a 50 kDa centrifugal concentrator to 10-16.5 mg/mL prior to being used for crystallization trials.

### Cell Lysate Substrate Specificity Assay

1.5 mL of auto-induction mediaa⁶⁵ containing 100 μg/mL carbenicillin was inoculated with 15 μL of overnight culture (LB media) of E. coli BL21(DE3) harboring the pET45-h6.AchP plasmid. The culture was incubated for 18 h at 37° C. then transferred to a 1.5 mL microfuge tube. The culture was centrifuged, media poured off and the cell pellets were resuspended in 250 μL of lysis buffer (50 mM HEPES pH 7.0, 1 mM EDTA, 0.5% Triton X-100, 4 mM MgSO₄, 50 mM NaCl, and 1 mg/mL lysozyme). The cell lysis mixture was incubated for 3 h at 37° C. then centrifuged again to collect the insoluble cell debris. The supernatant containing the soluble cell lysate was transferred to a new 1.5 mL microfuge tube. 10 μL of soluble cell lysate was transferred to a 96-well (Costar™ 96-well flat-bottom polystyrene) plate containing 20 μL lysis buffer and 170 μL substrate solution (20 mM MES pH 6.5, 200 mM sodium molybdate, 10 mM donor (Glc1-P or GlcNAc1-P) and acceptor (50 mM D-glucose (Glc), 50 mM glucosamine (GlcN), 20 mM chitobiose (GlcN-β1,4-GlcN), 50 mM N-acetylglucosamine (GlcNAc), 10 mM N,N′-diacetylchitobiose (GlcNAc-β1,4-GlcNAc), 50 mM D-galactose, 50 mM D-mannose (Man), 50 mM D-xylose (Xyl), 50 mM D-fructose (Fru), 50 mM RL-rhamnose (Rha), 20 mM gentiobiose (Gen), 50 mM maltose (Mal), 20 mM cellobiose (Cel), 10 mM laminaribiose (Lam), 5 mM sophorose (Sop) or 20 mM P-lactose (bLac)) then incubated for 3 h at 37° C. To initiate molybdenum blue formation, 50 μL of the substrate lysate mixture was transferred to a new 96-well color development plate followed by addition of 150 μL development solution (0.24% ascorbic acid and 0.25 N HCl). The color development plate was incubated for 5 min at room temperature before the reaction was stopped by adding 100 μL of quenching solution (3% acetic acid and 3% citrate). Absorbance at 655 nm was measured with a BioTek Synergy H1 Hybrid™ microtiter plate reader.

### Purified AchP Substrate Specificity Assay

Thirty-six donor and acceptor combinations were done with purified AchP in a 25 μL reaction volume. 10 μL buffer (200 mM HEPES pH 7.0, 200 mM NaCl, 10 mM MgSO₄, 400 mM sodium molybdate) was combined with 5 μL water, 2.5 μL donor (100 mM Glc1-P, 100 mM GlcNAc1-P or 100 mM GalNAc1-P) and 2.5 acceptor (500 mM Glc, 400 mM GlcN, 500 mM GlcNAc, 100 mM GlcNAc-β1-pNP, 500 mM Gal, 500 mM GalN, 500 mM GalNAc, 100 mM GalNAc-α1-pNP, 100 mM Glc-β1,3-Glc, 100 mM GlcNAc-β1,4-GlcNAc, 500 mM ManNAc or no acceptor) in a 200 μL PCR tube. 5 μL of purified AchP (0.2 mg/mL) was added to each reaction which were then incubated for 3 h at room temperature. Each 25 μL reaction was transferred to a separate well of a 96-well plate. 75 μL of development solution was added to each well then incubated for 5 min at room temperature before the reaction was stopped by adding 50 μL of quenching solution. Absorbance at 655 nm was measured with a BioTek Synergy H1 Hybrid™ microtiter plate reader.

### AchP Kinetics Analysis

Kinetic parameters for AchP reverse phosphorolysis were determined using the phosphate release method described previously⁴⁴. In brief, phosphate release was coupled to the formation of molybdenum blue, which can be quantified by measuring absorbance at 655 nm. Phosphate concentration was determined using a standard curve ranging between 0 and 10 mM phosphate. Kinetic parameters for AchP were determined with four donor and acceptor combinations: (A) Glc1-P and GlcNAc, (B) Glc1-P and GalNAc, (C) GlcNAc1-P and GlcNAc, and (D) GlcNAc1-P and GalNAc. Donor concentration was held constant at 10 mM while the concentrations of the acceptors were varied (as described below). Reactions were initiated by adding 5 μL of 0.25 mg/mL AchP to 10 μL 2× buffer (200 mM HEPES pH 7.0, 200 mM NaCl, 10 mM MgSO₄ and 400 mM sodium molybdate), 2.5 μL of 100 mM donor, 2.5 μL of lox acceptor and 5 μL water. Reactions were stopped at the times (t) indicated below by boiling for 5 min, then 20 μL from each reaction was transferred to a 96-well plate. Molybdenum blue formation was initiated by adding 90 μL of color development solution (0.1 N HCl and 0.24% sodium ascorbate) then incubated for 30 min at room temperature. The color development reaction was stopped by adding 90 μL of quenching solution (68 mM sodium citrate and 2% acetic acid). Absorbance at 655 nm was measured with a BioTek Synergy H1 Hybrid™ microtiter plate reader. All reactions were performed in triplicate and acceptor concentrations were chosen to encompass apparent Kₘ value where possible. (A) 10 mM Glc1-P; 0, 1, 5, 10, 25, 50, 100, 200 and 300 mM GlcNAc; t=2 min 45 s. (B) 10 mM Glc1-P; 0, 5, 25, 50, 100, 150, 200, 300 and 380 mM GalNAc; t=5 min. (C) 10 mM GlcNAc1-P; 0, 0.1, 0.25, 0.5, 1, 2, 5, 10, 25 and 50 mM GlcNAc; t=3 min. (D) 10 mM GlcNAc1-P; 0, 5, 10, 25, 50, 100, 150, 200 and 300 mM GalNAc; t=3 min. Non-linear regression was performed using GraphPad Prism™ version 6.0. For (A) and (C) data were fit using the Michaelis-Menten equation incorporating substrate inhibition:

$${v}_{0} = \frac{{V}_{max} [ S ]}{{K}_{m} + [ S ] ( \frac{1 + [ S ]}{{K}_{i}} )}$$

For (B) and (D) data were fit using the standard Michaelis-Menten equation:

$${v}_{0} = \frac{{V}_{max} [ S ]}{{K}_{m} + [ S ]}$$

### Oligomerization Analysis

Acholetin oligomerization was assayed with 10 mM Glc1-P, GlcNAc1-P or GalNAc1-P as donors and GlcNAc (FIG. 1C) or GalNAc (FIG. 6) as acceptor with donor/acceptor ratios of 1:10, 1:1 and 100:1. 0.5 μL of 100 mM donor was combined with 1 μL 5× acceptor (500 mM, 50 mM or 0.5 mM), 2.5 μL reaction buffer A (100 mM HEPES pH 7.0, 100 mM NaCl, 5 mM MgSO4) and 0.5 μL water. Reactions were initiated by adding 0.5 μL AchP (6 mg/mL) then incubating at room temperature for 3 h for those containing Glc1-P and GalNAc1-P and 30 min for those with GlcNAc1-P. Reactions were stopped by diluting 50× with super DHB MALDI matrix (Fluka™) then analyzed by MALDI-TOF MS (Bruker Autoflex™).

### Synthesis of GlNAc-β1,3-GlNAc

GlcNAc1-P and GlcNAc were dissolved in buffer (HEPES, pH 7.0), the enzyme AchP was added and the reaction mixture was incubated at 37° C. Final reaction conditions: GlcNAc1P (15 mg/mL) and GlcNAc (40 mg/mL), AchP (0.05 mg/mL), 50 mM HEPES. Reaction progress was monitored by TLC (BuOH:MeOH:NH₄OH:H₂O 5:4:4:2, p-anisaldehyde staining). After GlcNAc1-P was fully consumed, the enzyme was removed from the reaction mixture by ultrafiltration (MWCO 10 kDa, Satorius™). The product was isolated by gel filtration chromatography (eluent: H₂O; Bio-gel P2, BioRad™), fractions containing pure product were pooled and lyophilized. The pure dimer was characterized by MALDI-TOF MS (Bruker Autoflex™) and NMR (Bruker AV-400™ MHz spectrometer; solvent: D₂O).

### Synthesis of Glc-β1,3-GlcNAc-pNP

Glc1-P and GlcNAc were dissolved in buffer (HEPES, pH 7.0), AchP was added and the reaction mixture was incubated at 37° C. Final reaction conditions: 15 mg/mL Glc1-P and 40 mg/mL GlcNAc, 0.05 mg/mL AchP, 50 mM HEPES. Reaction progress was monitored by TLC (BuOH:MeOH:NH₄OH:H₂O 5:4:4:2, p-anisaldehyde staining). After Glc1-P was fully consumed, the enzyme was removed from the reaction mixture by ultrafiltration (MWCO 10 kDa, Satorius™) and product was isolated by gel filtration chromatography (eluent: H₂O; Bio-gel P2, BioRad™). The fraction containing pure product was lyophilized and characterized by MALDI-TOF MS (Bruker Autoflex™) and NMR (Bruker AV-400™ MHz spectrometer; solvent: D₂O).

### Core 3 Mucin-Like O-Glycan Analog Synthesis

AchP was used to synthesize T-antigen core 3 analogs, GlcNAc-β1,3-GalNAc-MU and GlcNAc-P1,3-GalNAc-pNP using GlcNAc1-P as donor and either 4-methylumbelliferyl N-acetyl-α-D-galactosaminide (GalNAc-MU) or 4-nitrophenyl N-acetyl-α-D-galactosaminide (GalNAc-pNP) as acceptor. For GlcNAc-β1,3-GalNAc-MU, 2 μL of 100 mM GlcNAc1-P (Carbosynth™) was combined with 1 μL 100 mM GalNAc-MU (prepared by Dr. Hongming Chen), 15 μL reaction buffer A (100 mM HEPES pH 7.0, 100 mM NaCl, 5 mM MgSO4). For GlcNAc-β1,3-GalNAc-pNP, 5 μL of 100 mM GlcNAc1-P was combined with 2.5 μL 100 mM GalNAc-pNP (prepared by Dr. Hongming Chen), 37.5 μL reaction buffer A. Reactions were initiated by addition of 2 μL (MU reactions) or 5 μL (pNP reactions) of AchP (6 mg/mL) and incubated at room temperature for 1 h. Reaction progress was monitored by TLC with a mobile phase of EtOAc:MeOH:H₂O 7:2:1 and visualized with molybdate TLC stain or under UV light (FIG. 7) and analyzed by mass spectrometry (FIG. 7). Both GlcNAc-β1,3-GalNAc-MU and GlcNAc-β1,3-GalNAc-pNP were assayed as potential substrates for the core 3 T-antigen cleaving enzyme endo-α-N-acetylgalactosaminidase from Streptococcus pneumoniae R6 (EngSP)^{47,66}. For GlcNAc-β1,3-GalNAc-MU, in a 96-well plate 2 μL of the AchP reaction was combined with 48 μL reaction buffer A and reaction initiated by adding 0.4 μL spGH101 (10 mg/mL). Release of MU was monitored continuously in a BioTek Synergy H1 Hybrid™ microtiter plate reader (Ex. 365 nm, Em. 440 nm) (FIG. 6). For GlcNAc-β1,3-GalNAc-pNP, in a 96-well plate 10 μL of the AchP reaction was combined with 39 μL reaction buffer A and initiated by adding 1 μL EngSP (10 mg/mL). Release of pNP was monitored continuously in a BioTek Synergy H1 Hybrid™ microtiter plate reader (absorbance 400 nm) (FIG. 6).

### Acholetin Two-Pot Large Scale Synthesis

### GlcNAc1-P Synthesis

In 60 mL, 1 g GlcNAc, 3 g ATP were dissolved in Tris, pH 7.0 buffer with MgCl₂. NahK was added and the reaction was incubated at 37° C. for 18 h (FIG. 2A). Final reaction conditions: GlcNAc (75 mM), ATP (100 mM), MgCl₂ (10 mM), NahK (0.08 mg/mL) and 967 mM Tris, pH 7.0. ATP, ADP, AMP and free phosphate was precipitated by the addition of barium acetate. 6.7 mL of 1 M barium acetate was added, the slurry was centrifuged, then the supernatant was transferred to clean 50 mL Falcon™ tubes. This barium acetate precipitation process was repeated twice more (FIG. 2A). GlcNAc1-P was precipitated from the supernatant by adding 267 mL of ethanol followed by centrifugation. The pellet was then washed with 333 mL acetone then dried under vacuum for 18 h (FIG. 2A). Total mass of the dried pellet was 2.66 g, which is composed primarily of GlcNAc1-P (maximum theoretical yield was 1.36 g) and barium acetate.

### Acholetin Synthesis

In 10 mL, 2.66 g of the GlcNAc1-P/barium acetate product was dissolved in 10 mM HEPES, pH 7.0 buffer with 0.4 mM GlcNAc. The donor/acceptor ratio was set roughly to 1000:1 to maximize the degree of polymerization (DP) of the acholetin product. AchP was added to a final concentration of 0.6 mg/mL to initiate the reaction. After addition of AchP, the reaction began to turn cloudy as the phosphate released from reverse phosphorolysis formed an insoluble barium salt. The reaction was incubated at room temperature for 48 h (FIG. 2B). The precipitated barium phosphate salt was removed by centrifugation, the supernatant was then incubated at 70° C. for 5 minutes to precipitate the AchP, which was also removed by centrifugation. The product was desalted on either a Sephadex G-25™ column with a bed height of 33 cm or a PD-10 Desalting Column (GE Healthcare™). The 33 cm column was attached to the AKTA (same as above) and equilibrated with degassed water. 4.4 mL of the acholetin mixture was injected onto the column with a 5 mL/min flowrate and the fraction collector set to collect 2 mL fractions after 20 mL post injection (FIG. 9). Fractions 4-13 were individually lyophilized. The combined mass was 151.5 mg which equates to a 344.3 mg yield from the whole 10 mL reaction. NMR analysis was performed on fraction 4, the fraction containing the highest proportion of high molecular weight acholetin oligomers (FIG. 9B). Fraction 4 was dissolved in D₂O and subjected to 1H, H-H COSY, HSQC and HMBC NMR analyses. The same sample was also reduced by NaBH₄ (followed by quenching with acid, 4× treatment with MeOH and removal of solvent) and subjected to the same NMR analyses to identify the reducing end GlcNAc unit. A second desalting preparation was done by applying 1 mL of the acholetin mixture to a PD-10 Desalting Column according to the manufacturer's instructions with water used as the mobile phase. The desalted 3.5 mL elution fraction was lyophilized then resuspended in water to a final concentration of 50 mg/mL. This material was analyzed by Multi-Angle Light Scattering (MALS).

### Nuclear Magnetic Resonance

### GlcNAc-β1,3-GlcNAc

¹H NMR (400 MHz, D₂O) δ 5.11 (d, J_{1α,2α}=3.4 Hz, 1H, H-1α), 4.66 (d, J_{1β,2β}=8.3 Hz, 1H, H-1_{β}), 4.58 (d, J_{1′(β,2′(β)}=8.3 Hz, 1H, H-1′_{(β)}), 4.57 (d, J_{1′(α),2′(α)}=8.2 Hz, 1H, H-1′_{(α)}), 3.95 (ddd, J_{1α,2α}=3.4, J_{2,NH}=10.5, J_{2,2}=10.5 Hz, 1H, H-2α), 3.96-3.86 (m, 4H, H-6 and H-6′), 3.93-3.87 (m, 1H, H-3α), 3.91-3.88 (m, 1H, H-2β), 3.91-3.85 (m, 1H, H-5α), 3.76-3.65 (m, 3H, H-2α, H-2′_{(α)} and H-2′_{(α)}), 3.75-3.72 (m, 1H, H-3β), 3.62-3.54 (m, 1H, H-3′), 3.59-3.50 (m, 1H, H-4α and H-4β), 3.51-3.43 (m, 3H, H-5β, H-5′_{(α)} and H-5′_{(β)}), 3.50-3.44 (m, 1H, H-4′), 2.07 (s, 3H, CH₃), 2.07 (s, 3H, CH₃), 2.02 (s, 3H, CH₃), 2.01 (s, 3H, CH₃).

¹³C NMR (101 MHz, D₂O) δ 174.43 (C═O), 174.40 (C═O), 174.00 (C═O), 173.72 (C═O), 101.13 (C-1′_{(β)}), 101.10 (C-1′_{(α)}), 95.00 (C-1β), 90.87 (C-1α), 81.41(C-3β), 78.86(C-3α), 75.76(C-5′_{(β)}), 75.69 (C-5′_{(α)}), 75.41 (C-5β), 73.26 (C-3′_{(α)}), 73.24 (C-3′_{(β)}), 71.09 (C-5α), 69.73 (C-4′), 68.50 (C-4α), 68.48(C- 4β), 60.74 (C-6β), 60.59 (C-6α), 60.53 (C-6′), 55.70 (C-2′_{(β)}), 55.67 (C-2′_{(α)}), 55.57 (C-2β), 52.97 (C-2α), 22.30 (CH₃), 22.24 (CH₃), 22.06 (CH₃).

β1,3 linkage was determined by HMBC {H-1′, C-3}.

### Glc-β1,3-GlcNAc-β1-pNP

¹H NMR (400 MHz, D₂O) δ 8.27 (d, J=9.3 Hz, 1H), 7.21 (d, J=9.3 Hz, 1H), 5.36 (d, J_{1,2}=8.5 Hz, 1H, H-1), 4.54 (d, J_{1′,2′}=7.8 Hz, 1H, H-1′), 4.19 (dd, J_{1,2}=8.5 Hz, J_{2,3}=10.4 Hz, 1H, H-2), 3.97 (dd, J_{5,6}=2.2 Hz, J_{6a,6b}=12.6 Hz, 1H, H-6a), 3.93 (dd, J_{2,3}=10.4 Hz, J_{3,4}=8.4 Hz, 1H, H-3), 3.92 (dd, J_{5′,6′α}=2.1 Hz, J_{6′a,6′b}=12.3 Hz, 1H, H-6′a), 3.83 (dd, J5,6b=5 Hz, J_{6a,6b}=12.6 Hz, 1H, H-6b), 3.77-3.71 (m, 1H, H-5), 3.74 (dd, J_{5′,6′b}=5.6 Hz, J_{6′a,6′b}=12.1 Hz, 1H, H-6′b), 3.68 (dd, J_{3,4}=8.4 Hz, J_{4,5}=9.7 Hz, 1H, H-4), 3.53-3.39 (m, 3H, H-3′, H-4′, H-5′), 3.33 (dd, J_{1′,2′}=7.8 Hz, J_{2′,3′}=9.3 Hz, 1H).

¹³C NMR (101 MHz, D₂O) δ 174.96, 161.62, 126.08, 116.52, 103.08 (C-1′), 98.35 (C-1), 82.03 (C-3), 75.95 (C-5), 75.80 (C-5′), 75.44 (C-3′), 72.87 (C-2′), 69.41 (C-4′), 68.22 (C-4), 60.58 (C-6′), 60.37 (C-6), 54.28 (C-2), 22.11 (CH₃).

β1,3 linkage was suggested by the 7-8 ppm downfield shift of the C-3 signal.

### Acholetin

*Non-reducing end unit is marked with′″. Unit next to non-reducing end is marked with”, all other units, except for the reducing end, are marked with’.

¹H NMR (400 MHz, D₂O) δ 5.32-5.28 (m, 1H, H-1a), 4.6-4.51 (m, all b-H-1's), 4.00-3.94 (H-2), 3.99-3.91 (H-3), 3.98-3.93 (m, H-5), 3.95-3.88 (m, H-6a, H-6′a, H-6″a, H-6′″a), 3.86-3.77 (m, H-3′), 3.86-3.79 (m, 1H, H-3″), 3.82-3.73 (m, H-6b, H-6′b, H-6″b, H6′″b), 3.76-3.67 (m, H-2′), 3.70 (dd, 1H, J=8.10 Hz, H-2′″), 3.59 (dd, 1H, J=8.10 Hz, H-3′″), 3.59-3.53 (m, 1H, H-4), 3.57-3.43 (m, H-2″, H-4′, H-4″, H-4′″, H-5′, H-5″, H-5′″).

¹³C NMR (75 MHz, D₂O) δ 174.69(C═O), 174.28(C═O), 174.05(C═O), 173.95(C═O), 101.82(C-1″), 101.14(C-1′″), 100-75(C-1′), 92.88(C-1α), 80.99(C-3″), 80.00(C-3′), 78.70(C-3_{α}), 75-93(C-5′″), 75.39(C-5′,C-5″), 73.37(C-3′″), 72.07(C-5_{α}), 69.97(C-4′″), 68.48(C-4_{α},C-4′,C-4″), 60.82(C-6,C-6′,C-6″,C-6′″), 58.67(C-2″), 55.84(C-2′″), 55.03(C-2′), 53.36(C-2_{α}), 22.63(CH₃), 22.45(CH₃), 20.23(CH₃).

β1,3 linkage was determined by HMBC {H-1, C-3′}, {H-1′, C-3′}, {H-1″, C-3′}, and {H-1′″, C-3″}

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

1. Song, E.-H., Shang, J. & Ratner, D. M. Polysaccharides. in Polymer Science: A Comprehensive Reference 9, 137-155 (Elsevier, 2012).
2. Smith, P. J. et al. Enzymatic Synthesis of Artificial Polysaccharides. ACS Sustain. Chem. Eng. 8, 11853-11871 (2020).
3. Seeberger, P. H. & Werz, D. B. Synthesis and medical applications of oligosaccharides. Nature 446, 1046-1051 (2007).
4. Yu, Y., Shen, M., Song, Q. & Xie, J. Biological activities and pharmaceutical applications of polysaccharide from natural resources: A review. Carbohydr. Polym. 183, 91-101 (2018).
5. Felt, O., Buri, P. & Gurny, R. Chitosan: A Unique Polysaccharide for Drug Delivery. Drug Dev. Ind. Pharm. 24, 979-993 (1998).
6. Zhang, N., Wardwell, P. R. & Bader, R. A. Polysaccharide-based micelles for drug delivery. Pharmaceutics 5, 329-352 (2013).
7. Debele, T. A., Mekuria, S. L. & Tsai, H. C. Polysaccharide based nanogels in the drug delivery system: Application as the carrier of pharmaceutical agents. Mater. Sci. Eng. C 68, 964-981 (2016).
8. Sinha, V. R. & Kumria, R. Polysaccharides in colon-specific drug delivery. Int. J. Pharm. 224, 19-38 (2001).
9. Liu, Z., Jiao, Y., Wang, Y., Zhou, C. & Zhang, Z. Polysaccharides-based nanoparticles as drug delivery systems. Adv. Drug Deliv. Rev. 60, 1650-1662 (2008).
10. Barclay, T. G., Day, C. M., Petrovsky, N. & Garg, S. Review of polysaccharide particle-based functional drug delivery. Carbohydr. Polym. 221, 94-112 (2019).
11. Saidin, N. M., Anuar, N. K. & Meor Mohd Affandi, M. M. R. Roles of Polysaccharides in Transdermal Drug Delivery System and Future Prospects. J. Appl. Pharm. Sci. 8, 141-157 (2018).
12. Hudak, J. E. & Bertozzi, C. R. Glycotherapy: New advances inspire a reemergence of glycans in medicine. Chem. Biol. 21, 16-37 (2014).
13. Yu, X., Marshall, M. J. E., Cragg, M. S. & Crispin, M. Improving Antibody-Based Cancer Therapeutics Through Glycan Engineering. BioDrugs 31, 151-166 (2017).
14. Yang, X. & Bartlett, M. G. Glycan analysis for protein therapeutics. J. Chromatogr. B Anal. Technol. Biomed. Life Sci. 1120, 29-40 (2019).
15. Zahedi, P., Rezaeian, I., Ranaei-Siadat, S.-O., Jafari, S.-H. & Supaphol, P. A review on wound dressings with an emphasis on electrospun nanofibrous polymeric bandages. Polym. Adv. Technol. 21, 77-95 (2010).
16. Firsatz, M. et al. Functionalization of bacterial cellulose wound dressings with the antimicrobial peptide epsilon-poly-L-Lysine. Biomed. Mater. 13, 025014 (2018).
17. Aduba, D. C. & Yang, H. Polysaccharide fabrication platforms and biocompatibility assessment as candidate wound dressing materials. Bioengineering 4, 1 (2017).
18. Wittaya-Areekul, S. & Prahsarn, C. Development and in vitro evaluation of chitosan-poly-saccharides composite wound dressings. Int. J. Pharm. 313, 123-128 (2006).
19. Naseri-Nosar, M. & Ziora, Z. M. Wound dressings from naturally-occurring polymers: A review on homopolysaccharide-based composites. Carbohydr. Polym. 189, 379-398 (2018).
20. Zhang, Y. S. et al. 3D Bioprinting for Tissue and Organ Fabrication. Ann. Biomed. Eng. 45, 148-163 (2017).
21. Shin, J. Y., Yeo, Y. H., Jeong, J. E., Park, S. A. & Park, W. H. Dual-crosslinked methylcellulose hydrogels for 3D bioprinting applications. Carbohydr. Polym. 238, 116192 (2020).
22. Zennifer, A., Senthilvelan, P., Sethuraman, S. & Sundaramurthi, D. Key advances of carboxymethyl cellulose in tissue engineering & 3D bioprinting applications. Carbohydr. Polym. 256, 117561 (2021).
23. Li, S. et al. Chitosans for tissue repair and organ three-dimensional (3D) bioprinting. Micromachines 10, 765 (2019).
24. Xu, J. et al. Advances in the research of bioinks based on natural collagen, polysaccharide and their derivatives for skin 3D bioprinting. Polymers (Basel). 12, 1237 (2020).
25. McCarthy, R. R., Ullah, M. W., Booth, P., Pei, E. & Yang, G. The use of bacterial polysaccharides in bioprinting. Biotechnol. Adv. 37, 107448 (2019).
26. Mohan, T., Maver, T., Štiglic, A. D., Stana-Kleinschek, K. & Kargl, R. 3D bioprinting of polysaccharides and their derivatives: From characterization to application. in Fundamental Biomaterials: Polymers 105-141 (Elsevier, 2018). doi:10.1016/B978-0-08-102194-1.00006-2
27. Xiao, R. & Grinstaff, M. W. Chemical synthesis of polysaccharides and polysaccharide mimetics. Prog. Polym. Sci. 74, 78-116 (2017).
28. Danby, P. M. & Withers, S. G. Advances in Enzymatic Glycoside Synthesis. ACS Chem. Biol. 11, 1784-1794 (2016).
29. Kadokawa, J. I. Precision polysaccharide synthesis catalyzed by enzymes. Chem. Rev. 111, 4308-4345 (2011).
30. Lombard, V., Golaconda Ramulu, H., Drula, E., Coutinho, P. M. & Henrissat, B. The carbohydrate-active enzymes database (CAZy) in 2013. Nucleic Acids Res. 42, D490-D495 (2014).
31. Nakai, H., Kitaoka, M., Svensson, B. & Ohtsubo, K. Recent development of phosphorylases possessing large potential for oligosaccharide synthesis. Curr. Opin. Chem. Biol. 17, 301-309 (2013).
32. O'Neill, E. C. & Field, R. A. Enzymatic synthesis using glycoside phosphorylases. Carbohydr. Res. 403, 23-37 (2015).
33. Puchart, V. Glycoside phosphorylases: Structure, catalytic properties and biotechnological potential. Biotechnol. Adv. 33, 261-276 (2015).
34. Awad, F. N. Glycoside phosphorylases for carbohydrate synthesis: An insight into the diversity and potentiality. Biocatal. Agric. Biotechnol. 31, 101886 (2021).
35. Zallot, R., Oberg, N. & Gerlt, J. A. The EFI Web Resource for Genomic Enzymology Tools: Leveraging Protein, Genome, and Metagenome Databases to Discover Novel Enzymes and Metabolic Pathways. Biochemistry 58, 4169-4182 (2019).
36. Zallot, R., Oberg, N. O. & Gerlt, J. A. ‘Democratized’ genomic enzymology web tools for functional assignment. Curr. Opin. Chem. Biol. 47, 77-85 (2018).
37. Gerlt, J. A. Genomic Enzymology: Web Tools for Leveraging Protein Family Sequence-Function Space and Genome Context to Discover Novel Functions. Biochemistry 56, 4293-4308 (2017).
38. Gerlt, J. A. et al. Enzyme function initiative-enzyme similarity tool (EFI-EST): A web tool for generating protein sequence similarity networks. Biochim. Biophys. Acta—Proteins Proteomics 1854, 1019-1037 (2015).
39. Macdonald, S. S. et al. Development and Application of a High-Throughput Functional Metagenomic Screen for Glycoside Phosphorylases. Cell Chem. Biol. 26, 1001-1012.e5 (2019).
40. Lazarev, V. N. et al. Complete genome and proteome of Acholeplasma laidlawii. J. Bacteriol. 193, 4943-4953 (2011).
41. De Groeve, M. R. M. et al. Development and application of a screening assay for glycoside phosphorylases. Anal. Biochem. 401, 162-167 (2010).
42. Hidaka, M. et al. Chitobiose phosphorylase from Vibrio proteolyticus, a member of glycosyl transferase family 36, has a clan GH-L-like (α/α)6 barrel fold. Structure 12, 937-947 (2004).
43. Park, J. K., Keyhani, N. O. & Roseman, S. Chitin Catabolism in the Marine Bacterium Vibrio furnissii. J. Biol. Chem. 275, 33077-33083 (2000).
44. Kuhaudomlarp, S. et al. Identification of Euglena gracilis β-1,3-glucan phosphorylase and establishment of a new glycoside hydrolase (GH) family GH149. J. Biol. Chem. 293, 2865-2876 (2018).
45. Petrović, D. M., Kok, I., Woortman, A. J. J., Ćirić, J. & Loos, K. Characterization of Oligocellulose Synthesized by Reverse Phosphorolysis Using Different Cellodextrin Phosphorylases. Anal. Chem. 87, 9639-9646 (2015).
46. Kudelka, M. R., Ju, T., Heimburg-Molinaro, J. & Cummings, R. D. Simple sugars to complex disease—mucin-type 0-glycans in cancer. Adv. Cancer Res. 126, 53-135 (2015).
47. Koutsioulis, D., Landry, D. & Guthrie, E. P. Novel endo-α-N-acetylgalactosaminidases with broader substrate specificity. Glycobiology 18, 799-805 (2008).
48. Cai, L. et al. A chemoenzymatic route to N-acetylglucosamine-1-phosphate analogues: substrate specificity investigations of N-acetylhexosamine 1-kinase. Chem. Commun. 2944 (2009). doi:10.1039/b904853g
49. Eddenden, A., Kitova, E. N., Klassen, J. S. & Nitz, M. An Inactive Dispersin B Probe for Monitoring PNAG Production in Biofilm Formation. ACS Chem. Biol. 15, 1204-1211 (2020).
50. Robbins, P. W., Overbye, K., Albright, C., Benfield, B. & Pero, J. Cloning and high-level expression of chitinase-encoding gene of Streptomyces plicatus. Gene 111, 69-76 (1992).
51. Mark, B. L. et al. Structural and Functional Characterization of Streptomyces plicatus β-N-Acetylhexosaminidase by Comparative Molecular Modeling and Site-directed Mutagenesis. J. Biol. Chem. 273, 19618-19624 (1998).
52. Williams, S. J., Mark, B. L., Vocadlo, D. J., James, M. N. G. & Withers, S. G. Aspartate 313 in the Streptomyces plicatusHexosaminidase Plays a Critical Role in Substrate-assisted Catalysis by Orienting the 2-Acetamido Group and Stabilizing the Transition State. J. Biol. Chem. 277, 40055-40065 (2002).
53. Laidlaw, P. P. & Elford, W. J. A new group of filterable organisms. Proc. R. Soc. London. Ser. B-Biol. Sci. 120, 292-303 (1936).
54. Windsor, H. M., Windsor, G. D. & Noordergraaf, J. H. The growth and long term survival of Acholeplasma laidlawii in media products used in biopharmaceutical manufacturing. Biologicals 38, 204-210 (2010).
55. Chernov, V. M. et al. Extracellular membrane vesicles secreted by mycoplasma Acholeplasma laidlawii PG8 are enriched in virulence proteins. J. Proteomics 110, 117-128 (2014).
56. Volokhov, D. V., Graham, L. J., Brorson, K. A. & Chizhikov, V. E. Mycoplasma testing of cell substrates and biologics: Review of alternative non-microbiological techniques. Mol. Cell. Probes 25, 69-77 (2011).
57. Chernov, V. M. et al. Extracellular membrane vesicles and phytopathogenicity of Acholeplasma laidlawii PG8. Sci. World J. 2012, 1-6 (2012).
58. Marcone, C. Molecular biology and pathogenicity of phytoplasmas. Ann. Appl. Biol. 165, 199-221 (2014).
59. Gilliam, J. M. & Morowitz, H. J. Characterization of the plasma membrane of Mycoplasma laidlawii. Biochim. Biophys. Acta-Biomembr. 274, 353-363 (1972).
60. Terry, T. M. & Zupnik, J. S. Weak association of glucosamine-containing polymer with the Acholeplasma Laidlawii membrane. BBA-Biomembr. 291, 144-148 (1973).
61. McElhaney, R. N. The structure and function of the Acholeplasma laidlawii plasma membrane. BBA-Rev. Biomembr. 779, 1-42 (1984).
62. Shannon, P. et al. Cytoscape: A software Environment for integrated models of biomolecular interaction networks. Genome Res. 13, 2498-2504 (2003).
63. Nishimoto, M. & Kitaoka, M. Identification of N-Acetylhexosamine 1-Kinase in the Complete Lacto-N-Biose I/Galacto-N-Biose Metabolic Pathway in Bifidobacterium longum. Appl. Environ. Microbiol. 73, 6444-6449 (2007).
64. Cai, L. et al. A chemoenzymatic route to N-acetylglucosamine-1-phosphate analogues: Substrate specificity investigations of N-acetylhexosamine 1-kinase. Chem. Commun. 2944-2946 (2009). doi:10.1039/b904853g
65. Studier, F. W. Protein production by auto-induction in high density shaking cultures. Protein Expr. Purif. 41, 207-234 (2005).
66. Willis, L. M., Zhang, R., Reid, A., Withers, S. G. & Wakarchuk, W. W. Mechanistic Investigation of the Endo-α-N-acetylgalactosaminidase from Streptococcus pneumoniae R6. Biochemistry 48, 10334-10341 (2009).
67. Computational Molecular Biology (Lesk, A. M., Ed.) Oxford University: NY (1988).
68. Biocomputing: Informatics and Genome Projects (Smith, D. W., Ed.) Academic: NY (1993).
69. Computer Analysis of Sequence Data, Part I (Griffin, A. M., and Griffin, H. G., Eds.) Humana: NJ (1994).
70. Sequence Analysis in Molecular Biology (von Heinje, G., Ed.) Academic (1987).
71. Sequence Analysis Primer (Gribskov, M. and Devereux, J., Eds.) Stockton: NY (1991).
72. Higgins and Sharp, CABIOS. 5:151-153 (1989).
73. Higgins, D. G. et al., Comput. Appl. Biosci., 8:189-191 (1992).
74. Thompson, J. D. et al., Nucleic Acids Research, 22 (22): 4673-4680 (1994).
75. Morando M. et al. Mimicking Chitin Chemical Synthesis Conformational Analysis and Molecular Recognition of the β(1→3)N-Acetylchitopentaose Analogue Chem. Eur. J. (2010) 16:4239-4249.
76. Kinzy, W. and Schmitt R. R. Liebigs Ann. Chem. (1985) 1537-1545.
77. Bradford, M. M. A Rapid and Sensitive Method for the Quantitation of Microgram Quantities of Protein Utilizing the Principle of Protein-Dye Binding. Analytical Biochemistry (1976) 72 (1-2), 248-254. https://doi.org/10.116/0003-2697(76)90527-3.

What is claimed is:

1. A polysaccharide, the polysaccharide comprising repeated monomers of N-acetyl-glucosamine (GlcNAc) or N-acetylgalactosamine (GalNAc) linked by glycosidic bonds having a β-configuration between the C1 position of the first GlcNAc or GalNAc ring and the C3 position of the adjacent GlcNAc or GalNAc ring, having the structure of Formula I 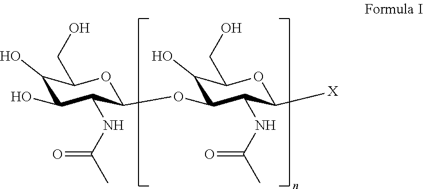
wherein,
n is an integer of 10 or greater; and
X is selected from —OH; and

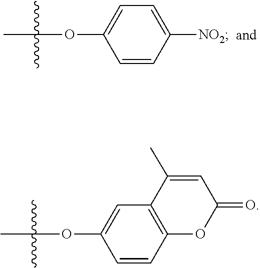 [SMILES: *Oc1ccc([N+](=O)[O-])cc1 ; "and" ; *Oc1ccc2oc(=O)cc(C)c2c1]

2. The polysaccharide of claim 1, wherein the hydroxyl groups at C4 are equatorial.

3. The polysaccharide of claim 1, wherein the hydroxyl groups at C4 are axial.

4. The polysaccharide of claims 1, 2, or 3, wherein n is between 10 and 3,000.

5. The polysaccharide of claims 1, 2, or 3, wherein n is between 10 and 1,000.

6. The polysaccharide of any one of claims 1-5, wherein X is —OH.

7. The polysaccharide of any one of claims 1-6, wherein polysaccharide is purified.

8. The polysaccharide of any one of claims 1-7, wherein the polysaccharide is lyophilized.

9. The polysaccharide of any one of claims 1-8, wherein polysaccharide forms part of a pharmaceutical composition, a cosmetic composition, a food composition, or a beverage composition.

10. The polysaccharide of any one of claims 1-9, wherein polysaccharide is for use as a part of a pharmaceutical composition, a cosmetic composition, a food composition, a beverage composition, a vaccine composition, or as a coating for a textile or as a coating for a medical device.

11. A method of making an oligosaccharide or a polysaccharide, the method comprising enzymatic synthesis with a glycoside phosphorylase (GP) that links monomeric GlcNAc or GalNAc via a β-1,3-glycosidic linkage, wherein the oligosaccharide or the polysaccharide comprises repeated monomers of GlcNAc or GalNAc linked by glycosidic bonds having a β-configuration between the C1 position of the first GlcNAc or GalNAc ring and the C3 position of the adjacent GlcNAc or GalNAc ring, having the structure of Formula I 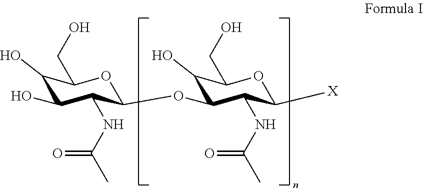
wherein,
n is an integer between 2 and 9, to form the oligosaccharide;
n is an integer of 10 or greater, to form the polysaccharide; and
X is selected from —OH;

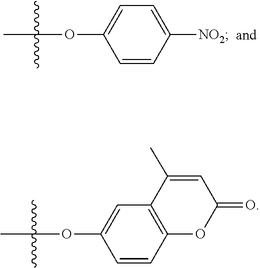 [SMILES: *Oc1ccc([N+](=O)[O-])cc1 ; "and" ; *Oc1ccc2oc(=O)cc(C)c2c1]

12. The method of claim 11, wherein the GP enzyme is a β-1,3-GlcNAc phosphorylase.

13. The method of claim 11 or 12, wherein the glycoside phosphorylase comprises an amino acid sequence that is at least 95% identical to SEQ ID NO: 2 and wherein the enzyme has β-1,3-GlcNAc phosphorylase enzyme activity.

14. The method of claim 11, 12, or 13, wherein the β-1,3-GlcNAc phosphorylase enzyme is the phosphorylase peptide comprising an amino acid sequence that is identical to SEQ ID NO: 2.

15. The method of any one of claims 11-14, wherein X is —OH.

16. A method of making an oligosaccharide or a polysaccharide, the method comprising:
(a) generating a GlcNAc-1-P or GalNAc-1-P, as a glycosyl donor, by reacting an N-acetylhexosamine-1-kinase (NahK) with GlcNAc or GalNAc and ATP;
(b) initiating a reverse phosphorolysis reaction by mixing the GlcNAc-1-P or GalNAc-1-P precipitate from step (a) with a glycosyl acceptor with a glycoside phosphorylase;
wherein the oligosaccharide or the polysaccharide comprises repeated monomers of GlcNAc or GalNAc linked by glycosidic bonds having a β-configuration between the C1 position of the first GlcNAc or GalNAc ring and the C3 position of the adjacent GlcNAc or GalNAc ring, having the structure of Formula II 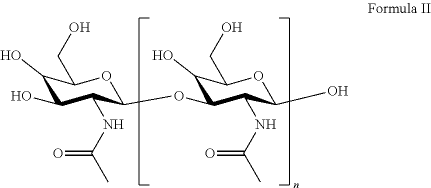
wherein,
n is an integer between 2 and 9, to form the oligosaccharide; and
n is an integer of 10 or greater, to form the polysaccharide.

17. The method of claim 16, further comprising:
(c) precipitating the oligosaccharide or polysaccharide product from the reaction mixture of step (b); and
(d) purifying the oligosaccharide or polysaccharide product from the reaction mixture of step (c).

18. The method of claim 17, wherein the method further comprises lyophilizing the oligosaccharide or polysaccharide product from the reaction mixture of step (d).

19. The method of claim 16, 17, or 18, wherein step (a) is carried out in a first reaction chamber and step (a) is carried out in a second reaction chamber.

20. The method of any one of claims 16-19, wherein step (a) is carried out for 18 h at 37° C. and where step (b) is carried out for 48 h at room temperature.

21. The method of any one of claims 16-20, wherein at molar ratio of GlcNAc or GalNAc:ATP is 1:1.3.

22. The method of any one of claims 16-21, wherein the NahK is isolated from Bifidobacterium longum comprising an amino acid sequence that is at least 95% identical to SEQ ID NO: 1 and wherein the enzyme has NahK enzyme activity.

23. The method of any one of claims 16-22, wherein the GlcNAc-1-P is alternatively produced by phosphorolysis of chitin or N,N-di-acetylchitobiose using a chitobiose phosphorylase or a chitinase.

24. The method of claim 23, wherein the chitobiose phosphorylase comprises an amino acid sequence that is at least 95% identical to SEQ ID NO: 4 or a chitinase comprises an amino acid sequence that is at least 95% identical to SEQ ID NO: 5

25. The method of any one of claims 16-24, wherein the glycosyl acceptor is GlcNAc.

26. The method of any one of claims 16-25, wherein the donor:acceptor ratio is at least 100:1.

27. The method of any one of claims 16-25, wherein the donor:acceptor ratio is at least 1000:1.

28. The method of any one of claims 16-25, further comprising continual removal of the phosphate from the reaction solution.

29. The method of claim 28, wherein the removal of the phosphate is by precipitation with a counter ion.

30. The method of claim 29, wherein the counter ion is barium acetate.

31. The method of any one of claims 16-30, wherein the glycoside phosphorylase is a β-1,3-glycoside phosphorylase.

32. The method of any one of claims 16-31, wherein the glycoside phosphorylase has binding sites specific for GlcNAc-1-P as a glycosyl donor and GlcNAc as a glycosyl acceptor.

33. The method of any one of claims 16-31, wherein the glycoside phosphorylase has binding sites specific for GalNAc-1-P as a glycosyl donor and GalNAc as a glycosyl acceptor.

34. The method of any one of claims 16-33, wherein the glycoside phosphorylase is a β-1,3-GlcNAc phosphorylase isolated from the mycobacterium Acholeplasma laidlawii.

35. The method of claim 34, wherein the β-1,3-GlcNAc phosphorylase enzyme is a phosphorylase peptide comprising an amino acid sequence that is at least 95% identical to SEQ ID NO:2.

36. The method of any one of claims 16-35, wherein the NahK enzyme is a peptide comprising an amino acid sequence that is at least 95% identical to SEQ ID NO:1.

37. A method of adding GlcNAc or GalNAc via a β-1,3 linkage to a GlcNAc or GalNAc at the non-reducing end of an oligosaccharide, a polysaccharide, a chitin or a chito-oligosaccharide, the method comprising reverse phosphorolysis with a glycoside phosphorylase.

38. The method of claim 37, wherein the donor:acceptor ratio is at least 100:1.

39. The method of claim 37 or 38, wherein the donor:acceptor ratio is at least 1000:1.

40. The method of claim 37, 38, or 39, wherein the glycoside phosphorylase is a β-1,3-GlcNAc phosphorylase.

41. The method of any one of claims 37-40, wherein the glycoside phosphorylase is a β-1,3-GlcNAc phosphorylase isolated from the mycobacterium Acholeplasma laidlawii.

42. The method of claim 41, wherein the β-1,3-GlcNAc phosphorylase enzyme is the phosphorylase peptide comprising an amino acid sequence that is at least 95% identical to SEQ ID NO:2.

43. The method of claim 41 or 42, wherein the β-1,3-GlcNAc phosphorylase enzyme is the phosphorylase peptide comprising an amino acid sequence that is identical to SEQ ID NO:2.

44. A reaction composition, the reaction composition comprising at least:
(a) a GlcNAc-1-P or GalNAc-1-P as a glycosyl donor;
(b) GlcNAc or GalNAc as a glycosyl acceptor; and
(c) a β-1,3-GlcNAc phosphorylase enzyme comprising an amino acid sequence that is at least 95% identical to SEQ ID NO: 2, wherein the amino acid sequence has β-1,3-GlcNAc phosphorylase enzyme activity, and wherein the β-1,3-GlcNAc phosphorylase enzyme synthesizes a β-1,3-glycosidic linkages between the donor and acceptor.

45. The reaction composition of claim 44, wherein there is a donor:acceptor ratio of at least 100:1.

46. The reaction composition of claim 44 or 45, wherein there is a donor:acceptor ratio of at least 1000:1.

47. The reaction composition of claim 44, 45 or 46, wherein the degree of polymerization (DP) is at least 6.

48. The reaction composition of claim 44, 45 or 46, wherein the degree of polymerization (DP) is at least 10.

49. The reaction composition of any one of claims 44-48, wherein the DP is at least about 10.

50. The reaction composition of any one of claims 44-49, wherein the acceptor is an oligosaccharide or a polysaccharide a non-reducing end of a GlcNAc or GalNAc.

51. The reaction composition of any one of claims 35-38, wherein the acceptor is a chitin or a chito-oligosaccharide or a chito-polysaccharide.

52. A method of making an polysaccharide of any one of claims 1-5, by enzymatic synthesis with a glycoside phosphorylases (GP) that links monomeric GlcNAc via a β-1,3-glycosidic linkage.

53. The method of claim 52, wherein the β-1,3-GlcNAc phosphorylase enzyme is the phosphorylase peptide comprising an amino acid sequence that is at least 95% identical to SEQ ID NO: 2 and wherein the enzyme has β-1,3-GlcNAc phosphorylase enzyme activity.

54. The method of claim 52 or 53, wherein the β-1,3-GlcNAc phosphorylase enzyme is the phosphorylase peptide comprising an amino acid sequence that is identical to SEQ ID NO: 2.


SEQUENCE LISTING (5 sequences)

SEQ ID NO: 1 - protein, 359 aa, Bifidobacterium longum
MTESNEVLFGIASHFALEGAVTGIEPYGDGHINTTYLVTTDGPRYILQQMNTSIFPDTVNLMRNVELVTSTLKAQGKETLDIVPTTSGATWAEIDGGAWRVYKFIEHTVSYNLVPNPDVFREAGSAFGDFQNFLSEFDASQLTETIAHFHDTPHRFEDFKAALAADKLGRAAACQPEIDFYLSHADQYAVVMDGLRDGSIPLRVTHNDTKLNNILMDATTGKARAIIDLDTIMPGSMLFDFGDSIRFGASTALEDEKDLSKVHFSTELFRAYTEGFVGELRGSITAREAELLPFSGNLLTMECGMRFLADYLEGDIYFATKYPEHNLVRTRTQIKLVQEMEQKASETRAIVADIMEAAR

SEQ ID NO: 2 - protein, 842 aa, Acholeplasma laidlawii
MKLKQDVISIYQKISLFESGQLNITKLASGAYYLDDELTLITDPVNSGARFPYAVNGMTIWAYASGYISINHSSYYILPPNLEGKEPFLDFFGIEQDGNNTYPVSLLGVSERNDEIENKRYTVFSKNIAYYITVTKNFLYAVTVYISKDFKIYFNTVAHNLTGETKQITLSSFFNMLFKYDSGESIETKWFKKVSYENNMFIYDAPEDIDRHTRIENYGVVKRHLHTKPKNIQNTTSRIDYVGKRYRSVRNALSIRSLKFEKAPLVTNFTDTAINADLINYEVKAYDTIISSYRIETCHDKDTLNKMMASDLTDKEIKKVYEGLSNTQSYDFDNFGISFKGVNDNRVDDKVLNQFLKLVNYQIHFSSLSSNSGTVFLGVRDVMQQLESSLIWDRKNVRSKILEVLSFIDPSGLPPRQYALPPKEGNPRMDLRPFIDQGLWIISTLHTYLAYTEDYDILNEVCGYYERIEPNSAKKSKVENSVLEHLIRVTNYLVSNIDPSTYGLKALYGDWNDALDGLGLIEGSSGYGNGVSVMATLQLYENLERMIEILKLVDPQNEHINTYEVVRHNLSLGINKYAVVIKQDEKRVLHGWGHDRSYFVGSFNDPDGHSRNSLTSNAFYIISDMIKNTPEMKPHLLHAFHNLDSKYGLKTFDPAMQDFHGFGRIINLPPGTAENAATYVHATLFGVLALYMLGEGDFANEQVLKVLPITKKEMSTSPFIMPNSYVHNEELNMDGESMSDWYTGSANTLLKTLIRGLFGLEVKFDHLRLRPSKAFFSKEATLMVSIGNKLTRIVYKNNNNGNRTFKLNGKVIEAKLDTLSGLLYIDINKSILEHQNVIHIQD

SEQ ID NO: 3 - DNA, 2529 bp, Acholeplasma laidlawii
atgaaacttaaacaagatgtaatcagtatttatcaaaaaatttcactttttgaaagtggccaattaaatattacaaagcttgctagtggtgcttactacttagatgatgaattaacacttataacagatcctgtcaatagtggagctaggtttccatatgccgtaaatggtatgaccatttgggcatatgcatcaggttatatttcaataaaccattcatcttactatatactaccgccaaatttagaaggtaaagaaccatttttagatttttttggaattgaacaagatggtaacaacacatatcctgtgtctttactaggtgtatcagagcgtaatgatgaaatagaaaataaacgttacacagtgtttagtaaaaatatagcttactatattacagttactaagaacttcttatatgcggttacagtctatattagtaaagactttaaaatttactttaatacagtagcacacaacctaacaggagagaccaaacaaattacactttcatcattctttaatatgctatttaaatatgatagcggtgaaagtattgaaacaaaatggtttaaaaaggtaagttatgaaaataacatgtttatatatgatgcaccagaagatattgatagacacaccagaattgaaaattatggtgtggttaaaagacatcttcatacaaaacctaaaaacattcaaaatacaacttcaagaattgattatgtaggtaaacgttatagatcagttcgtaatgctttaagcattcgaagtctaaaatttgaaaaagcaccccttgttacaaattttacagatactgcaattaatgctgatttaattaactacgaagtgaaagcctatgataccatcattagtagttaccgtattgaaacatgccacgataaagatacattaaataagatgatggcatctgatttaactgataaagaaattaaaaaagtatatgaaggtttatctaatactcaaagttatgattttgataactttggcatctcctttaaaggtgtaaatgacaatcgagtagatgataaagtcttaaatcagtttttaaagttagttaattaccaaatacacttctcatccctatcttcaaactcaggtaccgtatttttaggtgttagagatgttatgcaacaattagaatcatcactgatttgggatagaaaaaatgtaagaagtaaaatattagaagtcctttcctttattgatccatccggattaccgcctagacaatatgcgcttcctcctaaagagggtaatccgaggatggatttaagaccatttattgaccaaggtctatggattatttcaacccttcatacctatttagcatatactgaggattatgatatcttaaatgaagtatgtgggtattatgagcgtattgaaccaaacagtgctaaaaaatctaaagtagaaaactctgtattagaacacttaattagagttactaattacttagtgtctaatattgatccaagtacatatggcctaaaggcattatacggagactggaatgatgcacttgatggcttaggtttaattgaaggtagttcaggttatggtaatggtgtttctgttatggcaacactccaactatatgaaaacttagaacgcatgattgaaatcttaaaactagttgatcctcaaaatgaacatattaacacatatgaggtggtaagacataatttatctttaggcattaataaatacgcagtcgtaataaagcaagatgaaaaacgcgttttacatggttggggacatgacagaagttactttgtaggtagctttaatgacccggatggtcactcaagaaatagcttaacctctaatgctttttatatcatctcagatatgattaaaaatacacctgagatgaaaccacatttattacacgcattccataatcttgactctaaatatggtctaaaaacatttgatccagccatgcaagacttccacggatttggtcgtattataaacctaccacctggtacagctgaaaatgcagcaacttatgtacatgcaacactttttggtgtcttagcactttatatgttaggtgaaggagattttgctaatgagcaagtcttgaaagttttacctattactaaaaaggaaatgtctacatcaccatttattatgccaaactcatatgtacacaatgaggagcttaacatggacggtgagtccatgagtgactggtatacaggttctgcaaatacattacttaaaactttaattcgtggtttatttggccttgaagtaaagtttgatcacctaagacttcgtccatcaaaagcattcttctcaaaagaagcaacactgatggtaagtattggtaacaaacttacacgcatcgtgtataaaaacaataacaacggtaatagaacatttaaattaaatggtaaagtaatcgaagctaaactagatactttaagtggtctactatatatagatattaataaatccatattggaacaccaaaatgtaatacatatacaagactaa

SEQ ID NO: 4 - protein, 801 aa, Vibrio proteolyticus
MKYGYFDNDNREYVITRPDVPAPWTNYLGTEKFCTVISHNAGGYSFYNSPEYNRVTKFRPNATFDRPGHYVYLRDDDSGDYWSISWQPVAKSLDEAQYQIRHGLSYSKFQCDYNGIHARKTLFVPKGEDAEIWDVVIKNTSDQVRTISAFSFVEFSFSHIQSDNQNHQMSLYSAGTAYRPGLIEYDLYYNTDDFEGFYYLASTFDPDSYDGQRDRFLGLYRDEANPLAVEQGRCSNSAQTCYNHCGSLHKQFTLQPGEEIRFAYILGIGKGNGERLREHYQDVANIDAAFAAIKAHWDERCAKFQVKSPNQGLDTMINAWTLYQAETCVVWSRFASFIEVGGRTGLGYRDTAQDAISVPHANPEMTRKRIVDLLRGQVKAGYGLHLFDPDWFDPEKEDVAPSKSPTVVPTPSDEDKIHGIKDTCSDDHLWLIPTICKYVMETGETSFFDQMIPYADGGEASVYEHMKAALDFSAEYVGQTGICKGLRADWNDCLNLGGGESSMVSFLHFWALQEFIDLAKFLGKDQDVNTYTEMAANVREACETHLWDDEGGWYIRGLTKNGDKIGTAQQQEGRVHLESNTLAVLSGLASQERGEQAMDAVDEHLFSPYGLHLNAPSFSTPNDDIGFVTRVYQGVKENGAIFSHPNPWAWVAETKLGRGDRAMKFYDALNPYNQNDIIEKRIAEPYSYVQFIMGRDHQDHGRANHPWLTGTSGWAYFAVTNYILGVQSGFTGLSVDPCIPSDWPGFEVTRQWRGATYHIQVENPDHVSKGVKSITLNGAPIQGRIPPQAQGSDNQVVVVLG

SEQ ID NO: 5 - protein, 265 aa, Streptomyces griseus
ATCATAWSSSSVYTNGGTVSYNGRNYTAKWWTQNERPGTSDVWADKGACGTGGEGPGGNNGFVVSEAQFNQMFPNRNAFYTYKGLTDALSAYPAFAKTGSDEVKKREAAAFLANVSHETGGLFYIKEVNEANYPHYCDTTQSYGCPAGQAAYYGRGPIQLSWNFNYKAAGDALGINLLANPYLVEQDPAVAWKTGLWYWNSQNGPGTMTPHNAIVNNAGFGETIRSINGALECNGGNPAQVQSRINKFTQFTQILGTTTGPNLSC